(19) United States
(12) Patent Application Publication
(10) Pub. No.: US 2026/0335306 A1
(43) Pub. Date: Oct. 29, 2026

(54) DOSAGE REGIMENS OF ESTROGEN RECEPTOR DEGRADERS

(71) Applicants: Arvinas Operations, Inc., New Haven, CT (US); Pfizer Inc., New York, NY (US)

(72) Inventors: Ronald Peck, Cheshire, CT (US); Sheryl Maxine Gough, Startford, CT (US); John J. Flanagan, Guilford, CT (US); Sibyl Anderson, New York, NY (US); Ian Charles Anthony Taylor, Madison, CT (US); Julia Perkins Smith, New York, NY (US); Weiwei Tan, San Diego, CA (US)

(21) Appl. No.: 19/107,532

(22) PCT Filed: Aug. 30, 2023
(86) PCT No.: PCT/US2023/031574

§ 371 Date: Feb. 27, 2025

Related U.S. Application Data
(60) Provisional application No. 63/402,651, filed on Aug. 31, 2022.
(60) Provisional application No. 63/402,651, filed on Aug. 31, 2022.
(60) Provisional application No. 63/443,892, filed on Feb. 7, 2023.
(60) Provisional application No. 63/454,485, filed on Mar. 24, 2023.
(60) Provisional application No. 63/454,422, filed on Mar. 24, 2023.
(60) Provisional application No. 63/508,503, filed on Jun. 15, 2023.

(51) Int. Cl. A61K 31/496; A61K 31/454; A61K 31/506; A61K 31/519; A61P 35/00; A61P 35/04

(52) U.S. Cl. CPC A61K 31/496; A61K 31/454; A61K 31/506; A61K 31/519; A61P 35/00; A61P 35/04

(57) ABSTRACT

Disclosed herein are methods for treating cancer comprising administering to a subject a daily dose of Compound A, or a pharmaceutically acceptable salt thereof, in combination with a CDK4/6 inhibitor.

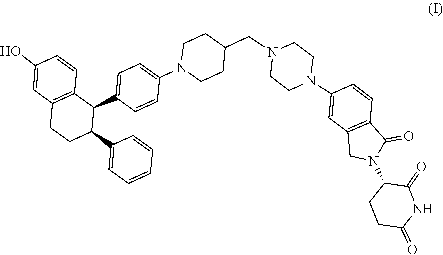 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

### CROSS-REFERENCES TO RELATED APPLICATIONS

This application claims priority to, and the benefit of, U.S. Application No. 63/402,651, filed Aug. 31, 2022, U.S. Application No. 63/443,892, filed Feb. 7, 2023, U.S. Application No. 63/454,485, filed Mar. 24, 2023, U.S. Application No. 63/454,422, filed Mar. 24, 2023, and U.S. Application No. 63/508,503, filed Jun. 15, 2023, the entirety of each of which is incorporated by reference herein.

### BACKGROUND OF THE INVENTION

Certain bifunctional compounds target specific cellular proteins for degradation via the ubiquitin-proteasome system. Examples of such proteolysis targeting chimeric compounds (i.e., “PROTAC® protein degraders”) that target the Estrogen Receptor (ER) for ubiquitination and subsequent degradation are disclosed in International Publication No. WO 2018/102725, which is incorporated herein by reference in its entirety. Such bifunctional molecules exhibit a range of pharmacological activities consistent with the degradation of the ER including, but not limited to, treatment or amelioration of a disease condition such as cancer (e.g., breast cancer, uterine cancer, ovarian cancer, prostate cancer, endometrial cancer), or endometriosis.

A bifunctional molecule of particular interest is vepdegestrant (i.e., (S)-3-(5-(4-((1-(4-((1R,2S)-6-hydroxy-2-phenyl-1,2,3,4-tetrahydronaphthalen-1-yl)phenyl) piperidin-4-yl)methyl) piperazin-1-yl)-1-oxoisoindolin-2-yl)piperidine-2,6-dione or (3S)-3-[1,3-dihydro-1-oxo-5-[4-[[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (referred to herein as “Compound A” or “Cpd A”)), which has the molecular formula of C₄₅H₄₉N₅O₄ and the following structure:

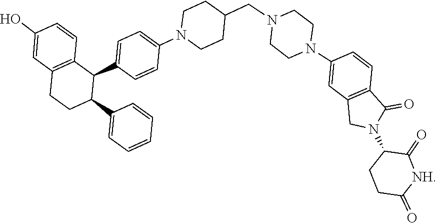 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is under development as a PROTAC® protein degrader that targets estrogen receptor (ER) for the potential treatment of breast cancer and has been shown to be a useful modulator of targeted protein ubiquitination and degradation via the ubiquitin-proteasome pathway.

There is a need for appropriate dosage regimens of Compound A as an oral therapy for treating cancers (e.g., breast cancer), to improve its benefit, including safety and efficacy, and convenience to patients while at the same time minimizing adverse events and risks to patients.

### SUMMARY OF THE INVENTION

The present disclosure provides, in part, dosage regimens for administering Compound A, or a pharmaceutically acceptable salt thereof, to a subject in combination therapies, for treating cancer. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Provided herein are methods for treating cancer comprising administering to a subject a daily dose of Compound A having a structure of:

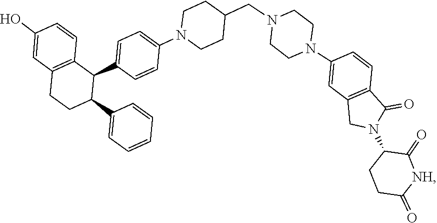 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in combination with a CDK4/6 inhibitor, wherein the daily dose of Compound A is about 100 mg or about 200 mg.

Provided herein are methods for treating cancer comprising administering to a subject a daily dose of Compound A having a structure of:

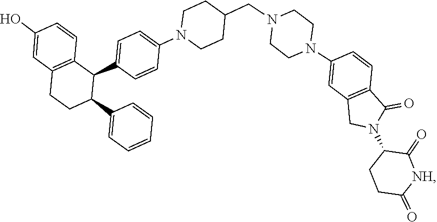 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

in combination with a CDK4/6 inhibitor.

In embodiments, the daily dose of Compound A is about 200 mg. In embodiments, the daily dose of Compound A is about 100 mg.

In embodiments, the CDK4/6 inhibitor is dalpiciclib, trilaciclib, lerociclib, AT7519M, dinaciclib, ribociclib, abemaciclib, or palbociclib, or a pharmaceutically acceptable salt thereof.

In embodiments, the CDK4/6 inhibitor is abemaciclib, ribociclib or palbociclib, or a pharmaceutically acceptable salt thereof. In embodiments, the CDK4/6 inhibitor is abemaciclib, or a pharmaceutically acceptable salt thereof. In embodiments, the CDK4/6 inhibitor is ribociclib, or a pharmaceutically acceptable salt thereof. In embodiments, the CDK4/6 inhibitor is palbociclib, or a pharmaceutically acceptable salt thereof.

In embodiments, Compound A, or a pharmaceutically acceptable salt thereof, may be administered daily in 28-day cycles in combination with the CDK4/6 inhibitor. In embodiments, Compound A may be administered daily in 28-day cycles in combination with the CDK4/6 inhibitor. In certain embodiments, the CDK4/6 inhibitor is palbociclib, or a pharmaceutically acceptable salt thereof. For example, palbociclib may be administered orally once a day at 125 mg/day for 21 days followed by 7 days off treatment for each 28-day cycle.

In embodiments, the daily dose of Compound A is administered once per day (QD).

In embodiments, the daily dose of Compound A is administered orally to the subject.

In embodiments, the subject is in a fed state.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer. For example, the breast cancer may be metastatic or locally advanced. Alternatively, the breast cancer may be estrogen receptor positive (ER+) breast cancer (e.g., human epidermal growth factor receptor 2 negative (HER2−)).

In embodiments, the subject is human.

It is to be understood that both the foregoing general description and the following detailed description are exemplary and explanatory only and are not restrictive of the invention, as claimed.

### BRIEF DESCRIPTION OF THE DRAWINGS

FIGS. 1A-1H are graphs showing quantification of the MCF7 and T47D live-cell imaging proliferation assays. FIGS. 1A and 1C show relative cell growth kinetics over 120-hours by live cell imaging of MCF7 and T47D cells dosed with Compound A (10 nM) or fulvestrant (1 nM), alone or in combination with abemaciclib (40 nM) at their respective approximate GI₅₀ concentrations. Each plot is representative of three independent experiments. FIGS. 1E and 1G show relative cell growth kinetics over 120-hours by live cell imaging of MCF7 and T47D cells dosed with Compound A (10 nM) and/or ribociclib (40 nM) at their respective approximate GI₅₀ concentrations. Each plot is representative of three independent experiments, except for the T47D fulvestrant arms in FIGS. 1A and 1C, which are the result of 2 independent experiments). FIGS. 1B, 1D, 1F and 1H show differences in relative MCF7 and T47D cell growth compared to vehicle control at 120-hours of treatment. Graphs show the mean of three independent experiments, except for the T47D fulvestrant arms in FIGS. 1B and 1D, which are the result of 2 independent experiments). Error bars=standard error of the mean (SEM). *p<0.025, **p≤0.008, ***p≤0.0006, ****p<0.0001, ns=not significant (one-way ANOVA test).

FIGS. 2A-2L are graphs showing viability and synergistic analysis of MCF7 cells at day-5 dosed with Compound A in combination with abemaciclib or ribociclib in an 8×8 block matrix. FIGS. 2A, 2B, 2G, and 2H show single agent curves of Compound A, abemaciclib, Compound A, and ribociclib, respectively. FIG. 2C shows Compound A dose-response shift with the addition of abemaciclib. FIG. 2I shows Compound A dose-response shift with the addition of ribociclib. Drug synergies were evaluated using Combenefit software. BLISS (2D and 2J), Loewe (2E and 2K) and Highest Single Agent (2F and 2L) model output is shown (representative of three independent experiments).

FIGS. 3A and 3B are graphs showing in-vivo efficacy studies of Compound A in combination with the CDK4/6 inhibitors abemaciclib (FIG. 3A) and ribociclib (FIG. 3B) using MCF7 orthotopic xenograft models. Mean tumor volumes are reported ±SEM. Single-day dosing holidays are indicated by small black arrows.

FIG. 4A is a graph showing body weights (±SEM) of MCF7 orthotopic xenograft efficacy with Compound A in combination with the CDK4/6 inhibitor abemaciclib. Compounds dosed as single agents or in combination, 10 mice/arm.

FIG. 4B is a graph showing body weights (±SEM) of MCF7 orthotopic xenograft efficacy with Compound A in combination with the CDK4/6 inhibitor ribociclib. Compounds dosed as single agents or in combination, 10 mice/arm.

FIG. 5 is a graph showing inhibition of tumor growth by Compound A (30 mg/kg, per os [oral dosing; PO]; once daily [QD]×28) or fulvestrant (200 mg/kg, subcutaneously; twice per week for 2 weeks followed by once-weekly for 2 weeks) as single agents or in combination with palbociclib (‘Palbo’; 60 mg/kg, PO; QD×28), a CDK4/6 inhibitor. Female severe combined immunodeficient mouse in non-obese background (NOD/scid) mice were implanted with MCF7 cells and compound administration was initiated once the tumors reached 200 mm³. Tumor volumes were evaluated twice per week for 28 days. At the end of the study, single-agent Compound A and fulvestrant inhibited tumor growth by 105% and 46%, respectively. When combined with palbociclib, the growth inhibition with Compound Aor fulvestrant was 131% and 108%, respectively. Data are presented as mean±standard error of the mean (SEM)

### DETAILED DESCRIPTION OF THE INVENTION

The present invention may be understood more readily by reference to the following detailed description of the embodiments of the invention and the Examples included herein. It is to be also understood that the terminology used herein is for the purpose of describing specific embodiments only and is not intended to be limiting.

Vepdegestrant (i.e., (S)-3-(5-(4-((1-(4-((1R,2S)-6-hydroxy-2-phenyl-1,2,3,4-tetrahydronaphthalen-1-yl)phenyl) piperidin-4-yl)methyl) piperazin-1-yl)-1-oxoisoindolin-2-yl) piperidine-2,6-dione or (3S)-3-[1,3-dihydro-1-oxo-5-[4-[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (also referred to herein as “Compound A”)):

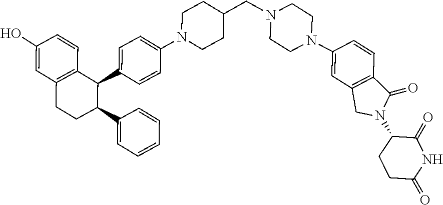 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is under development as a PROTAC® protein degrader that targets estrogen receptor (ER) for the potential treatment of breast cancer and has been shown to be a useful modulator of targeted protein ubiquitination and degradation via the ubiquitin-proteasome pathway.

Compound A and pharmaceutically acceptable salts thereof are disclosed in International Publication No. WO 2018/102725 and U.S. Pat. Nos. 10,647,698, 10,899,742 and 11,104,666; International Publication No. WO 2021/041348; U.S. Ser. No. 17/472,847; U.S. Ser. No. 17/548,842; and U.S. Ser. No. 17/873,748. The contents of each of the foregoing references are incorporated herein by reference in their entirety.

Cyclin-dependent kinases (CDKs) and related serine/threonine protein kinases are important cellular enzymes that perform essential functions in regulating eukaryotic cell division and proliferation. CDK catalytic units are activated by regulatory subunits known as cyclins. At least sixteen mammalian cyclins have been identified (Johnson D G, Walker C L. Cyclins and Cell Cycle Checkpoints. Annu. Rev. Pharmacol. Toxicol. (1999) 39:295-312). Cyclin B/CDK1, cyclin A/CDK2, cyclin E/CDK2, cyclin D/CDK4, cyclin D/CDK6, and likely other heterodynes are important regulators of cell cycle progression. Additional functions of cyclin/CDK heterodynes include regulation of transcription, DNA repair, differentiation, and apoptosis (Morgan D O, Cyclin-dependent kinases: engines, clocks, and microprocessors. Annu. Rev. Cell. Dev. Biol. (1997) 13:261-291).

CDK inhibitors have been demonstrated to be useful in treating cancer. Increased activity or temporally abnormal activation of cyclin-dependent kinases has been shown to result in the development of human tumors, and human tumor development is commonly associated with alterations in either the CDK proteins themselves or their regulators (Cordon-Cardo C. Mutations of cell cycle regulators: biological and clinical implications for human neoplasia. Am. J. Pathol. (1995) 147:545-560; Karp J E, Broder S. Molecular foundations of cancer: new targets for intervention. Nat. Med. (1995) 1:309-320; Hall M, Peters G. Genetic alterations of cyclins, cyclin-dependent kinases, and Cdk inhibitors in human cancer. Adv. Cancer Res. (1996) 68:67-108).

CDK4 and CDK6 are important regulators of cell cycle progression at the G1-S checkpoint, which are controlled by D-type cyclins and INK4 endogenous CDK inhibitors, such as p16^{INK4a} (CDKN2A). Dysregulation of the cyclin D-CDK4/6-INK4-retinoblastoma (Rb) pathway has been reported to be associated with development of endocrine therapy resistance.

Clinical trials for the CDK4/6 inhibitors palbociclib, ribociclib, and abemaciclib are ongoing for breast and other cancers, as single agents or in combination with other therapeutics. The use of CDK4/6 inhibitors in combination with endocrine therapy has demonstrated significant efficacy in the treatment of hormone receptor (HR)-positive, human epidermal growth factor 2 (HER2)-negative advanced or metastatic breast cancers, and CDK4/6 inhibitors, including palbociclib, ribociclib, and abemaciclib, have been approved in combination with endocrine therapy in a first- or second-line setting. Palbociclib, ribociclib, and abemaciclib have been approved for treatment of hormone receptor (HR)-positive, human epidermal growth factor receptor 2 (HER2)-negative advanced or metastatic breast cancer in combination with aromatase inhibitors, such as letrozole, in a first line setting and with fulvestrant in second or later lines of therapy in certain patients. (O'Leary et al. Treating cancer with selective CDK4/6 inhibitors. Nature Reviews (2016) 13:417-30).

Palbociclib, or 6-acetyl-8-cyclopentyl-5-methyl-2-(5-piperazin-1-yl-pyridin-2-ylamino)-8H-pyrido[2,3-d]pyrimidin-7-one (also referred to as “PD-0332991”) is a potent and selective inhibitor of CDK4 and CDK6, having the structure:

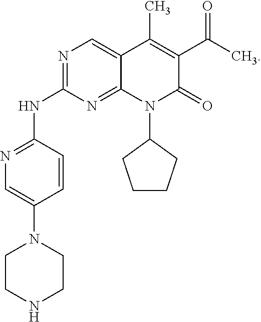 [SMILES: CC(=O)c1c(C)c2cnc(Nc3ccc(N4CCNCC4)cn3)nc2n(C2CCCC2)c1=O]

Palbociclib is described in WHO Drug Information, Vol. 27, No. 2, page 172 (2013). Palbociclib and pharmaceutically acceptable salts thereof are disclosed in International Publication No. WO 2003/062236 and U.S. Pat. Nos. 6,936,612, 7,456,168 and RE47,739; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588. The contents of each of the foregoing references are incorporated herein by reference in their entirety.

Abemaciclib, N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine, (e.g., as sold under the brand name Verzenio® (and others)) is a selective inhibitor of CDK4 and CDK6, having the structure:

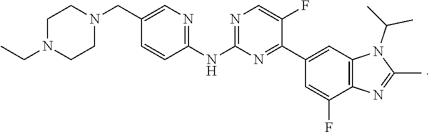 [SMILES: CCN1CCN(Cc2ccc(Nc3ncc(F)c(-c4cc(F)c5nc(C)n(C(C)C)c5c4)n3)nc2)CC1]

Abemaciclib and pharmaceutically acceptable salts thereof are disclosed in International Publication No. WO2010/075074 and U.S. Pat. No. 7,855,211. The contents of each of the foregoing references are incorporated herein by reference in their entirety.

Ribociclib, 7-cyclopentyl-N,N-dimethyl-2-[(5-piperazin-1-ylpyridin-2-yl)amino]pyrrolo[2,3-d]pyrimidine-6-carboxamide (e.g., as sold under the brand names Kisqali® and Kryxana®) is an inhibitor of cyclin D1/CDK4 and CDK6, having the structure:

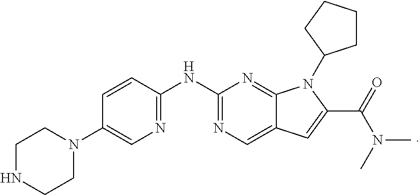 [SMILES: CN(C)C(=O)c1cc2cnc(Nc3ccc(N4CCNCC4)cn3)nc2n1C1CCCC1]

Ribociclib and pharmaceutically acceptable salts thereof are disclosed in International Publication Nos. WO2007140222, WO2010/020675, WO2012/064805 andWO2016/166703 and in U.S. Pat. Nos. 8,324,225, 8,415,355, 8,685,980, 8,962,630, 9,193,732, 9,416,136, 9,868,739, and 10,799,506. The contents of each of the foregoing references are incorporated herein by reference in their entirety.

While CDK4/6 inhibitors have shown significant clinical efficacy in ER-positive metastatic breast cancer, as with other kinases their effects may be limited over time by the development of primary or acquired resistance. The selective CDK4/6 inhibitor palbociclib has proven to be clinically efficacious in breast cancer (DeMichele A, Clark A S, Tan K S, et al. CDK4/6 inhibitor palbociclib (PD-0332991) in Rb+ advanced breast cancer: phase II activity, safety, and predictive biomarker assessment. Clin Cancer Res 2015; 21 (5): 995-1001; Finn R S, Martin M, Rugo H S, et al. Palbociclib and Letrozole in Advanced Breast Cancer. New Engl J Med 2016; 375 (20): 1925-36; Cristofanilli M, Turner N C, Bondarenko I, et al. Fulvestrant plus palbociclib versus fulvestrant plus placebo for treatment of hormone-receptor-positive, HER2-negative metastatic breast cancer that progressed on previous endocrine therapy (PALOMA-3): final analysis of the multicentre, double-blind, phase 3 randomised controlled trial. Lancet Oncol 2016; 17 (4): 425-39), however, after initial clinical benefit, acquired resistance to palbociclib may occur (Knudsen Erik S., Witkiewicz Agnieszka K., The Strange Case of CDK4/6 Inhibitors: Mechanisms, Resistance, and Combination Strategies. Trends Cancer (2017) 3 (1): 39-55).

### Definitions

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

As used herein, the singular form “a,” “an,” and “the” include plural references unless indicated otherwise. For example, “a” substituent includes one or more substituents.

As used herein, the term “about” when used to modify a numerically defined parameter (e.g., the dose of Compound A) means that the parameter may vary by as much as 10% below or above the stated numerical value for that parameter. For example, a dose of about 5 mg means 5 mg+10%, i.e., it may vary from 4.5 mg to 5.5 mg.

As used herein, terms, including, but not limited to, “agent,” “composition,” “compound,” “drug,” and “therapeutic agent” may be used interchangeably to refer to compounds included in the methods and uses of the present disclosure.

As used herein, the terms, “subject,” “participant,” and “patient,” are used interchangeably, to refer to any animal, including mammals. Mammals according to the disclosure include canine, feline, bovine, caprine, equine, ovine, porcine, rodents, lagomorphs, primates, humans, and the like, and encompass mammals in utero. In embodiments, humans are suitable subjects. Human subjects may be of any gender and at any stage of development.

Vepdegestrant (i.e., (S)-3-(5-(4-((1-(4-((1R,2S)-6-hydroxy-2-phenyl-1,2,3,4-tetrahydronaphthalen-1-yl)phenyl) piperidin-4-yl)methyl) piperazin-1-yl)-1-oxoisoindolin-2-yl) piperidine-2,6-dione or (3S)-3-[1,3-dihydro-1-oxo-5-[4-[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (also referred to herein as “Compound A”)) is a compound having the structure:

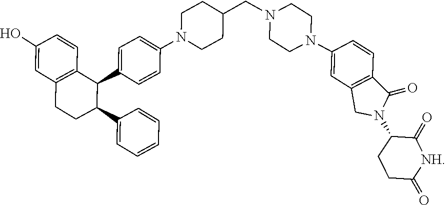 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is a Biopharmaceutics Classification System Class IV compound (low solubility/low permeability) Compound A can interconvert to its enimer Compound B:

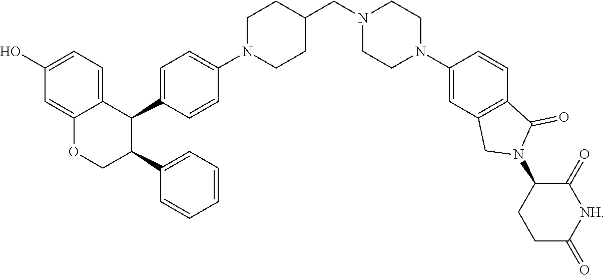 [SMILES: O=C1CC[C@@H](N2Cc3cc(N4CCN(CC5CCN(c6ccc([C@@H]7c8ccc(O)cc8OC[C@@H]7c7ccccc7)cc6)CC5)CC4)ccc3C2=O)C(=O)N1]

Without wishing to be bound by theory, preclinical data indicates that the exposure of Compound B is limited compared to Compound A (<26%). Evidence indicates that Compound B does not degrade the ER; however, Compound B shows similar antagonism of ER-dependent transcription compared to Compound A.

Cyclin-dependent kinases (CDKs) and related serine/threonine kinases are important cellular enzymes that perform essential functions in regulating cell division and proliferation. CDK inhibitors include Pan-CDK inhibitors that target a broad spectrum of CDKs or selective CDK inhibitors that target specific CDK(s).

Examples of CDK4/6 inhibitors include, but are not limited to, abemaciclib, ribociclib, and palbociclib. Additional examples of CDK4/6 inhibitors include lerociclib (also known as G1T38) and trilaciclib (also known as GTI128).

In embodiments, a CDK4/6 inhibitor of the present invention includes palbociclib. Unless otherwise indicated herein, palbociclib (also referred to herein as “palbo” or “Palbo”) refers to 6-acetyl-8-cyclopentyl-5-methyl-2-(5-piperazin-1-yl-pyridin-2-ylamino)-8H-pyrido[2,3-d]pyrimidin-7-one:

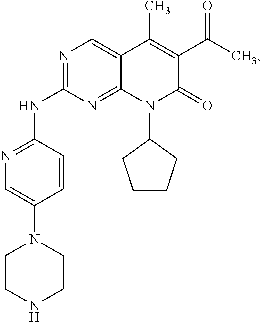 [SMILES: CC(=O)c1c(C)c2cnc(Nc3ccc(N4CCNCC4)cn3)nc2n(C2CCCC2)c1=O]

or a pharmaceutically acceptable salt thereof.

Alternatively, in embodiments, the CDK4/6 inhibitor is abemaciclib or ribociclib. Unless indicated otherwise, abemaciclib refers to N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine:

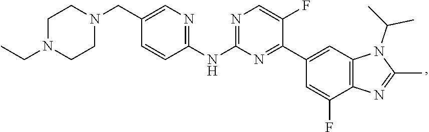 [SMILES: CCN1CCN(Cc2ccc(Nc3ncc(F)c(-c4cc(F)c5nc(C)n(C(C)C)c5c4)n3)nc2)CC1]

and ribociclib refers to 7-cyclopentyl-N,N-dimethyl-2-[(5-piperazin-1-ylpyridin-2-yl)amino]pyrrolo[2,3-d]pyrimidine-6-carboxamide:

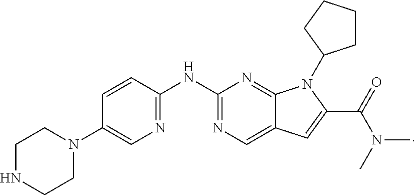 [SMILES: CN(C)C(=O)c1cc2cnc(Nc3ccc(N4CCNCC4)cn3)nc2n1C1CCCC1]

Other embodiments relate to the pharmaceutically acceptable salts of the compounds described herein. Pharmaceutically acceptable salts of the compounds described herein include the acid addition and base addition salts thereof.

Other embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases that form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine, and zinc salts.

The compounds described herein that are basic in nature can form a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)]salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate, and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

### Dosage Regimens

Provided herein are methods for treating cancer comprising administering to a subject a daily dose of Compound A having a structure of:

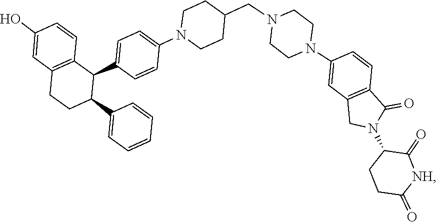 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in combination with a CDK4/6 inhibitor.

Also provided herein are methods for treating cancer comprising administering to a subject a daily dose of Compound A having a structure of:

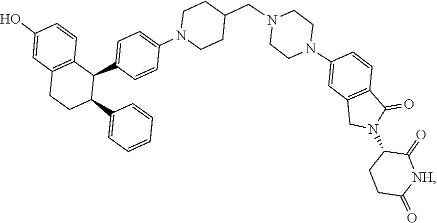 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

in combination with a CDK4/6 inhibitor.

In embodiments, the daily dose of Compound A having the structure:

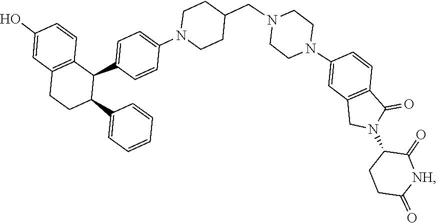 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is administered once per day (QD).

In embodiments, the daily dose of Compound A having the structure:

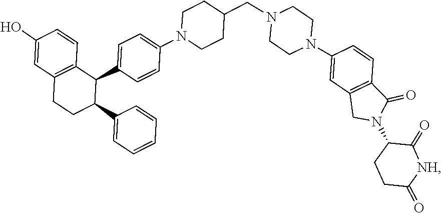 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is administered orally to the subject.

In embodiments, the subject is in a fed state.

In embodiments, the daily dose of Compound A:

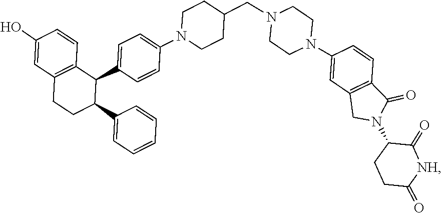 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is about 200 mg, or an equivalent amount of a pharmaceutically acceptable salt thereof.

In embodiments, the daily dose of Compound A:

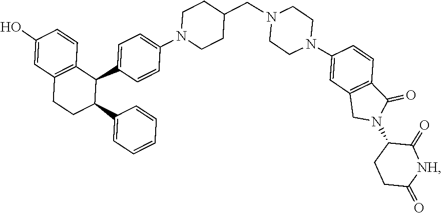 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is 200 mg or an equivalent amount of a pharmaceutically acceptable salt thereof.

In embodiments, the daily dose of Compound A:

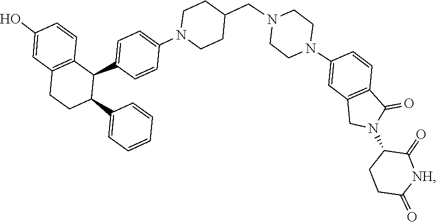 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is about 200 mg.

In embodiments, the daily dose of Compound A:

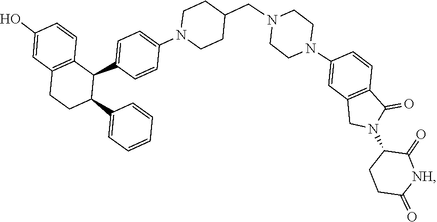 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is 200 mg.

In embodiments, the daily dose of Compound A:

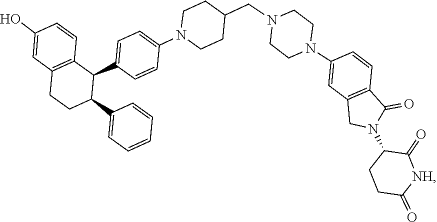 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is about 100 mg or an equivalent amount of a pharmaceutically acceptable salt thereof.

In embodiments, the daily dose of Compound A:

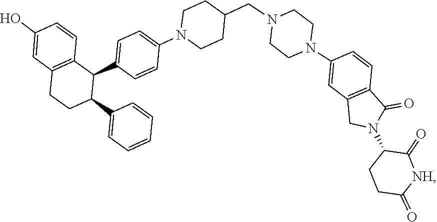 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, is 100 mg or an equivalent amount of a pharmaceutically acceptable salt thereof.

In embodiments, the daily dose of Compound A:

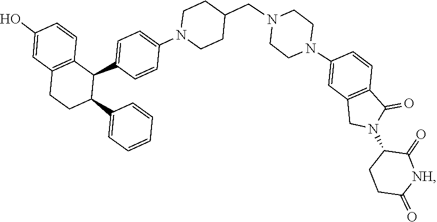 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is about 100 mg.

In embodiments, the daily dose of Compound A:

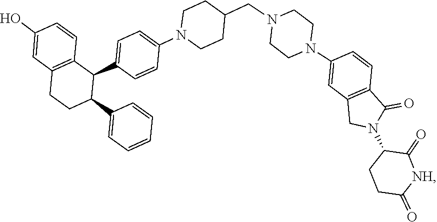 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is 100 mg.

In embodiments, Compound A:

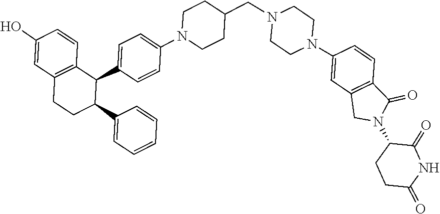 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is administered as a free base.

In embodiments, the CDK4/6 inhibitor is dalpiciclib, trilaciclib, lerociclib, AT7519M, dinaciclib, ribociclib, abemaciclib, or palbociclib, or a pharmaceutically acceptable salt thereof. In embodiments, the CDK4/6 inhibitor is dalpiciclib, trilaciclib, lerociclib, AT7519M, dinaciclib, ribociclib, abemaciclib, or palbociclib.

In embodiments, wherein the CDK4/6 inhibitor is abemaciclib, ribociclib, or palbociclib, or a pharmaceutically acceptable salt thereof. In embodiments, wherein the CDK4/6 inhibitor is abemaciclib, ribociclib, or palbociclib.

In embodiments, the CDK4/6 inhibitor is abemaciclib, or a pharmaceutically acceptable salt thereof. In embodiments, the CDK4/6 inhibitor is abemaciclib.

In embodiments, the CDK4/6 inhibitor is ribociclib, or a pharmaceutically acceptable salt thereof. In embodiments, the CDK4/6 inhibitor is ribociclib.

In embodiments, the CDK4/6 inhibitor is palbociclib, or a pharmaceutically acceptable salt thereof. In embodiments, the CDK4/6 inhibitor is palbociclib.

In embodiments, Compound A is administered daily in 28-day cycles. In certain embodiments, palbociclib, or a pharmaceutically acceptable salt thereof, is administered orally once a day at 125 mg/day for 21 days followed by 7 days off treatment for each 28-day cycle.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic or locally advanced.

In embodiments, the breast cancer is estrogen receptor positive (ER+) breast cancer.

In embodiments, the estrogen receptor positive (ER+) breast cancer is human epidermal growth factor receptor 2 negative (HER2−).

In embodiments, the subject is human.

Also disclosed herein Compound A:

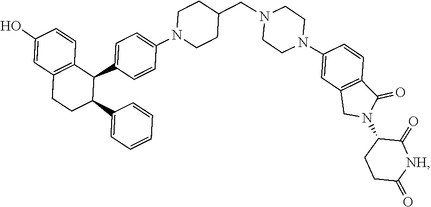 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, for use according to any one of foregoing embodiments.

Also disclosed herein Compound A is:

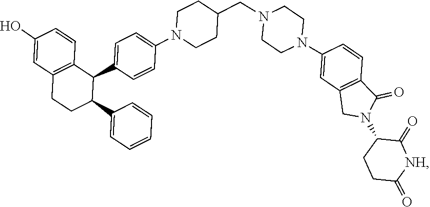 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, for use according to any one of foregoing embodiments.

Also disclosed herein are uses of Compound A:

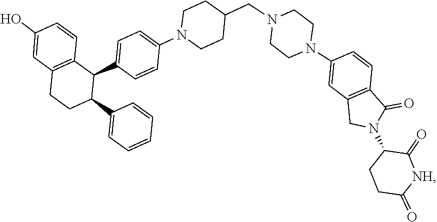 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in the manufacture of a medicament according to any one of foregoing embodiments.

Also disclosed herein are uses of Compound A:

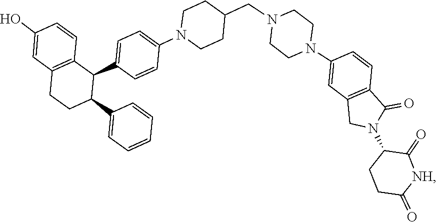 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, in the manufacture of a medicament according to any one of foregoing embodiments.

Each of the embodiments described herein may be combined with any other embodiment(s) described herein not inconsistent with the embodiment(s) with which it is combined.

### Administration and Dosing

The terms “treat” and “treating” a cancer or a cancer-associated disease, as used herein, mean to administer a combination therapy according to the present disclosure to a subject, participant or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The terms “treatment” and “therapy,” as used herein, unless otherwise indicated, refer to the act of treating as “treating” is defined immediately above. For the purposes of this disclosure, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. (2009) 50: 1S-10S).

“Fed condition” or “fed state” as used to describe a subject herein, means that the subject has eaten less than 4 hours before a time point of interest, such as the time of administering Compound A. In embodiments, a subject in the fed state has eaten within any of 4, 3, 2, 1, or 0.5 hours prior to administration of Compound A.

An “amount” for use and for treating a subject refers to an amount that provides, in single or multiple doses, in combination with one or more other agents, a detectable response of any duration of time (transient, medium, or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences, or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an agent in combination with one or more other agents, effective for producing a desired therapeutic effect upon administration to a subject, for example, to stem the growth, or result in the shrinkage, of a cancerous tumor. In reference to the treatment of cancer, a therapeutically effective amount refers to that amount that has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens also may be characterized as the ability to induce, enhance, maintain, or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

Embodiments of the present invention provide a dose, dosage, and dosing regimen comprising administering to a subject an amount, or an effective amount of Compound A, or a pharmaceutically acceptable salt thereof. The amount, or the therapeutically effective amount, can be a daily dose of about 200 mg. In another embodiment, a daily dose is 200 mg.

In embodiments, the daily dose of Compound A, or a pharmaceutically acceptable salt thereof, is administered once per day (QD).

The compounds disclosed herein may be administered orally. Oral administration may involve swallowing, so that the compound enters the gastrointestinal tract, or buccal or sublingual administration may be employed by which the compound enters the bloodstream directly from the mouth.

In embodiments, the daily dose of Compound A, or a pharmaceutically acceptable salt thereof, is administered orally.

Compound A, or a pharmaceutically acceptable salt thereof, may be present in a pharmaceutical composition, which includes a pharmaceutically acceptable excipient. A “pharmaceutically acceptable excipient” refers to a component that may be included in the compositions described herein, is physiologically suitable for pharmaceutical use, and causes no significant adverse effects nor therapeutic effects to a subject. The term “excipient” is used herein to describe any ingredient other than the compound(s) of the invention. The choice of excipient will to a large extent depend on factors such as the mode of administration, the effect of the excipient on solubility and stability, and the nature of the dosage form.

The compounds of the methods, uses, or combinations of the present invention may be formulated prior to administration. The formulation preferably will be adapted to the particular mode of administration. These compounds may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous, and nonaqueous oral solutions and suspensions, packaged in containers adapted for subdivision into individual doses.

In embodiments, palbociclib, or a pharmaceutically acceptable salt thereof, is administered at a daily dosage of about 125 mg once daily, about 100 mg once daily, about 75 mg once daily, about 50 mg daily, or about 25 mg daily. In embodiments, palbociclib, or a pharmaceutically acceptable salt thereof, is administered at a daily dosage of about 125 mg once a day. For example, palbociclib, or a pharmaceutically acceptable salt thereof, is administered at a dose of about 100 mg once daily, about 75 mg once daily, or about 50 mg once daily. In an embodiment, palbociclib, or a pharmaceutically acceptable salt thereof, is administered at a dose of about 100 mg once daily. In embodiments, palbociclib, or a pharmaceutically acceptable salt thereof, is administered at a dose of about 75 mg once daily. In embodiments, palbociclib, or a pharmaceutically acceptable salt thereof, is administered at a dose of about 50 mg once daily. Dosage amounts, provided herein, refer to the dose of the free base form of palbociclib, or are calculated as the free base equivalent of an administered palbociclib salt form. For example, a dosage or amount of palbociclib, such as 100 mg, 75 mg, or 50 mg, refers to the free base equivalent.

In embodiments, abemaciclib, or a pharmaceutically acceptable salt thereof, is administered at a daily dosage of about 400 mg (e.g., about 200 mg twice daily), about 300 mg (e.g., about 150 mg twice daily), about 200 mg (e.g., about 100 mg twice daily), or about 100 mg (e.g., about 50 mg twice daily). In embodiments, abemaciclib, or a pharmaceutically acceptable salt thereof, is administered at a daily dosage of about 300 mg (e.g., about 150 mg twice daily), about 200 mg (e.g., about 100 mg twice daily), or about 100 mg (e.g., about 50 mg twice daily). In embodiments, abemaciclib, or a pharmaceutically acceptable salt thereof, is administered at a daily dosage of 300 mg (e.g., about 150 mg twice daily). In an embodiment, abemaciclib, or a pharmaceutically acceptable salt thereof, is administered at a dose of about 200 mg (e.g., about 100 mg twice daily). In embodiments, abemaciclib, or a pharmaceutically acceptable salt thereof, is administered at a dose of about 50 mg twice daily about 100 mg (e.g., about 50 mg twice daily). Dosage amounts, provided herein, refer to the dose of the free base form of abemaciclib, or are calculated as the free base equivalent of an administered abemaciclib salt form. For example, a dosage or amount of abemaciclib, such as 200 mg, 150 mg, 100 mg, or 50 mg, refers to the free base equivalent.

In embodiments, ribociclib, or a pharmaceutically acceptable salt thereof, is administered at a daily dosage of about 600 mg once daily, about 400 mg once daily, about 200 mg once daily, or about 50 mg once daily. In embodiments, ribociclib, or a pharmaceutically acceptable salt thereof, is administered at a daily dosage of about 600 mg once daily. In an embodiment, ribociclib, or a pharmaceutically acceptable salt thereof, is administered at a dose of about 400 mg once daily. In embodiments, ribociclib, or a pharmaceutically acceptable salt thereof, is administered at a dose of about 200 mg once daily. Dosage amounts, provided herein, refer to the dose of the free base form of ribociclib, or are calculated as the free base equivalent of an administered ribociclib salt form. For example, a dosage or amount of ribociclib, such as 600 mg, 400 mg, 200 mg, refers to the free base equivalent.

Repetition of the administration or dosing regimens may be conducted as necessary to achieve the desired reduction or diminution of cancer cells. A “continuous dosing schedule,” as used herein, is an administration or dosing regimen without dose interruptions, e.g., without days off treatment. Repetition of 28-day treatment cycles without dose interruptions between the treatment cycles is an example of a continuous dosing schedule. In embodiments, the compounds of the combination of the present invention can be administered in a continuous dosing schedule. In embodiments, the compounds of the combination disclosed herein can be administered concurrently in a continuous dosing schedule.

In embodiments, Compound A is administered once daily to comprise a complete cycle of 28-days. Repetition of 28-day treatment cycles is continued during treatment in accordance with the methods and uses of the present disclosure.

The standard recommended dosing regimen, which includes the standard dosing schedule, for palbociclib, or a pharmaceutically acceptable salt thereof, is administration once daily for 21 consecutive days followed by 7 days off treatment to comprise a complete cycle of 28-days. Repetition of the 28-day cycles is continued during treatment with the combination of the present invention.

The standard clinical dosing regimen, for palbociclib, or a pharmaceutically acceptable salt thereof, is administration of 125 mg once daily for 21 consecutive days followed by 7 days off treatment to comprise a complete cycle of 28-days. Repetition of the 28-day cycles is continued during treatment with the combination of the present invention.

The standard clinical dosing regimen, for ribociclib, or a pharmaceutically acceptable salt thereof, is administration of 600 mg once daily for 21 consecutive days followed by 7 days off treatment to comprise a complete cycle of 28-days. Repetition of the 28-day cycles is continued during treatment with the combination of the present invention.

Also disclosed herein are kits comprising the therapeutic agents of the combination of the present disclosure and written instructions for administration of the therapeutic agents. In embodiments, the written instructions elaborate and qualify the modes of administration of the therapeutic agents, for example, for simultaneous or sequential administration of the therapeutic agents of the present disclosure. In embodiments, the written instructions elaborate and qualify the modes of administration of the therapeutic agents, for example, by specifying the days of administration for each of the therapeutic agents during a 28-day treatment cycle.

### Methods of Treatment

In embodiments, provided herein are methods for treating cancer in a subject comprising administering to the subject an effective amount of Compound A as described herein in combination with an amount of a CDK4/6 inhibitor.

The term “combination,” as used herein, unless otherwise indicated, refers to the use of Compound A with one or more therapeutic agents, wherein Compound A and the one or more therapeutic agents are administered intermittently, concurrently, or sequentially, according to the same or different route of administration and according to the same or different dosage schedules.

The term “locally advanced,” as used herein, as it relates to cancer, may or may not be treated with curative intent. For example, locally advanced breast cancer (LABC) is defined by the U.S. National Comprehensive Cancer Network as a subset of breast cancer characterized by the most advanced breast tumors in the absence of distant metastasis, wherein the tumors are more than 5 cm in size with regional lymphadenopathy; tumors of any size with direct extension to the chest wall or skin, or both (including ulcer or satellite nodules), regardless of regional lymphadenopathy; presence of regional lymphadenopathy (clinically fixed or matted axillary lymph nodes, or any of infraclavicular, supraclavicular, or internal mammary lymphadenopathy) regardless of tumor stage. (Garg et al. Curr Oncol. 2015 October; 22 (5): e409-e410; National Comprehensive Cancer Network NCCN Clinical Practice Guidelines in Oncology: Breast Cancer. Fort Washington, PA: NCCN; 2015. Ver. 2.2015.)

The term “metastatic” as used herein, as it relates to cancer, cannot be treated with curative intent. For example, metastatic breast cancer refers to breast cancer that has spread beyond the breast and nearby lymph nodes to other parts of the body, e.g., bones, liver, lungs, brain. (www.cancer.org/cancer/breast-cancer.)

Those skilled in the art will be able to recognize and diagnose locally advanced and metastatic cancer in a patient or subject.

For convenience, certain well-known abbreviations may be used herein, including: castration resistant prostate cancer (CRPC), estrogen receptor positive (ER+), human epidermal growth factor receptor 2 negative (HER2−), hormone receptor (HR), human epidermal growth factor receptor 2 positive (HER2+), non-small cell lung cancer (NSCLC), and progesterone receptor (PR).

In embodiments, the cancer is selected from lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, head and neck cancer, and combinations of two or more of the foregoing cancers.

Also disclosed herein are methods of treating cancer in a subject. In embodiments, the methods comprise treating cancer in a subject comprising administering to the subject an amount of the compounds described herein that are effective in treating the cancer.

In embodiments, the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the cancer is breast cancer, lung cancer, prostate cancer, pancreatic cancer, or ovarian cancer.

In embodiments, the cancer is breast cancer, lung cancer, or prostate cancer.

In embodiments, the cancer is breast cancer.

In embodiments, the breast cancer is metastatic breast cancer.

In embodiments, the breast cancer is locally advanced breast cancer.

In embodiments, the breast cancer is HR+ breast cancer.

In embodiments, the HR+ breast cancer is PR+ and/or ER+ breast cancer.

In embodiments, the breast cancer is PR+ breast cancer.

In embodiments, the breast cancer is ER+ breast cancer.

In embodiments, the breast cancer is ER+ HER2-breast cancer.

In embodiments, the breast cancer is ER+ HER2+ breast cancer.

In embodiments, the breast cancer is locally advanced or metastatic ER+ breast cancer.

In embodiments, the breast cancer is locally advanced or metastatic ER+ HER2-breast cancer. In embodiments, the breast cancer is locally advanced or metastatic ER+ HER2+ breast cancer.

In embodiments, the breast cancer is metastatic, ER+, HER2-breast cancer.

In embodiments, the breast cancer is metastatic, ER+, HER2-breast cancer that is also locally advanced.

In embodiments, the lung cancer is non-small cell lung cancer.

In embodiments, the lung cancer is locally advanced or metastatic non-small cell lung cancer.

In embodiments, the prostate cancer is CRPC.

In embodiments, the prostate cancer is locally advanced or metastatic CRPC.

Also disclosed herein are methods of treating solid tumors in a subject. In embodiments, disclosed herein are methods of treating solid tumors in a subject comprising administering to the subject an amount of the compounds described herein that are effective in treating the solid tumor.

In embodiments, the solid tumor is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

In embodiments, the solid tumor is breast cancer, lung cancer, prostate cancer, pancreatic cancer, or ovarian cancer.

In embodiments, the solid tumor is breast cancer, lung cancer, or prostate cancer.

In embodiments, the solid tumor is breast cancer. For example, in certain embodiments the breast cancer is HR+ breast cancer. In other embodiments, the HR+ breast cancer is PR+ and/or ER+ breast cancer ER+ breast cancer.

In embodiments, the solid tumor is breast cancer. For example, in certain embodiments, the breast cancer is ER+ HER2-breast cancer.

In embodiments, the solid tumor is breast cancer. For example, in certain embodiments, the breast cancer is ER+ HER2+ breast cancer.

In embodiments, the solid tumor is breast cancer. For example, in certain embodiments, the breast cancer is locally advanced or metastatic ER+ HER2-breast cancer.

In embodiments, the solid tumor is breast cancer. For example, in certain embodiments, the breast cancer is locally advanced or metastatic ER+ HER2+ breast cancer.

In embodiments, the solid tumor is lung cancer. For example, in certain embodiments, the lung cancer is non-small cell lung cancer.

In embodiments, the solid tumor is lung cancer. For example, in certain embodiments, the lung cancer is locally advanced or metastatic non-small cell lung cancer.

In embodiments, the solid tumor is prostate cancer. For example, in certain embodiments, the prostate cancer is CRPC.

In embodiments, the solid tumor is prostate cancer. For example, in certain embodiments, the prostate cancer is locally advanced or metastatic castration resistant prostate cancer.

Also disclosed herein are methods of treating hematologic tumors in a subject. In certain embodiments, the method comprises treating hematologic tumors in a subject comprising administering to the subject an amount of the compounds described herein that is effective in treating the hematologic tumor.

In embodiments, the hematologic tumor is leukemia, lymphoma, or multiple myeloma.

In embodiments, the hematologic tumor is leukemia or lymphoma.

Also disclosed herein are methods of treating cancer in a subject with locally advanced or metastatic ER+ HER2-breast cancer, CRPC, or NSCLC whose disease progressed on or is intolerant to standard therapy.

Also disclosed herein are methods of treating cancer in a subject with locally advanced or metastatic ER+ HER2-breast cancer, CRPC, or NSCLC whose disease progressed on or is intolerant to standard therapy.

Also disclosed herein are methods of treating cancer in a subject with locally advanced or metastatic 2L+ER+ HER2 breast cancer who have received prior hormonal/endocrine therapy and chemotherapy in the locally advanced/metastatic setting. In embodiments, the method comprises administering Compound A in combination with a CDK4/6 inhibitor to the subject.

Also disclosed herein are methods of treating cancer in a subject with locally advanced or metastatic 2L+ER+ HER2 breast cancer who have received prior treatment with a CDK4/6 inhibitor. In embodiments, the method comprises administering Compound A in combination with a CDK4/6 inhibitor to the subject.

Also disclosed herein are therapeutic combinations comprising:
(i) Compound A:

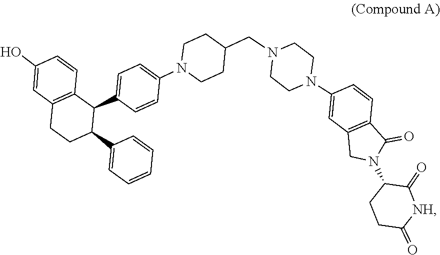 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof; and
(ii) a CDK4/6 inhibitor;
for simultaneous, separate, or sequential use in a method of treating cancer; wherein the method comprises administering a daily dose of Compound A, or a pharmaceutically acceptable salt thereof.

Also disclosed herein are therapeutic combinations comprising:
(i) Compound A:

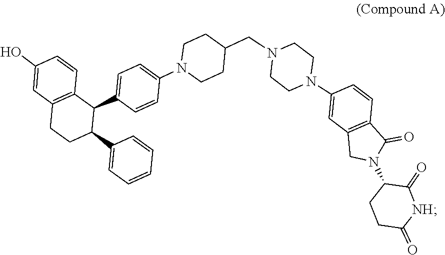 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

and
(ii) a CDK4/6 inhibitor;
for simultaneous, separate or sequential use in a method of treating cancer; wherein the method comprises administering a daily dose of Compound A.

Also disclosed herein is Compound A:

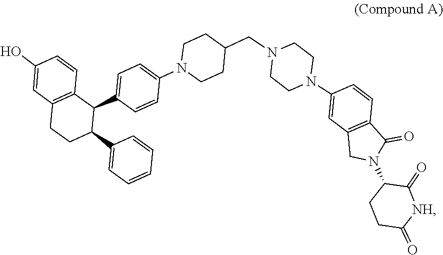 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof, for use in a method of treating cancer wherein the method comprises administering Compound A, or a pharmaceutically acceptable salt thereof, and wherein the method further comprises administering a CDK4/6 inhibitor.

Also disclosed herein is Compound A:

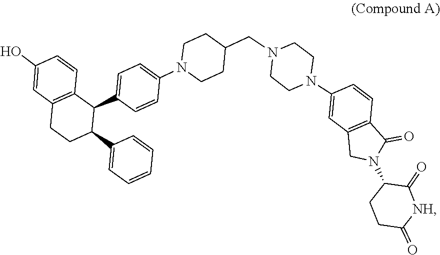 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

for use in a method of treating cancer wherein the method comprises administering Compound A, and wherein the method further comprises administering a CDK4/6 inhibitor.

Also disclosed herein is a CDK4/6 inhibitor for use in a method of treating cancer, wherein the method further comprises administering Compound A, or a pharmaceutically acceptable salt thereof.

Also disclosed herein is a CDK4/6 inhibitor for use in a method of treating cancer, wherein the method further comprises administering Compound A.

In embodiments, the method comprises administering a daily dose of about 200 mg of Compound A, or an equivalent amount of a pharmaceutically acceptable salt thereof. In embodiments, the method comprises administering a daily dose of about 200 mg of Compound A. In embodiments, the method comprises administering a daily dose of about 100 mg of Compound A.

### EXAMPLES

In order that this invention may be better understood, the following examples are set forth. These examples are for purposes of illustration only and are not to be construed as limiting the scope of the invention in any manner.

Abbreviations used herein include:
AUC₀₋₈=area under the plasma concentration-time curve from zero to 8 hours;
AUC₀₋₁₂=area under the plasma concentration-time curve from 0 to 12 hours;
AUCₜₐᵤ=area under the plasma concentration-time curve over the dosing interval, where tau=24 hours for once daily dosing and 12 hours for twice daily dosing;
BID=twice daily;
BMI=body mass index;
CI=confidence interval;
CR=complete response
CBR=clinical benefit rate;
Cₘₐₓ=maximum plasma concentration;
DDI=drug-drug interaction;
ER=estrogen receptor;
GCV=geometric coefficient of variation;
HER2=human epidermal growth factor receptor 2;
FIH=first-in-human;
IR=immediate-release;
mBC=metastatic breast cancer;
MTD=maximum tolerated dose;
ORR=overall response rate;
PPI=proton pump inhibitor;
PR=partial response
rBA=relative bioavailability;
RP2D=recommended Phase 2 dose;
QD=once daily;
SA=single agent;
SD=stable disease; and
TRAE=treatment-related adverse event.

### Compound a Clinical Trials (“the FIH Study” and “the Clinical Pharmacology Study”)

### The First-In-Human (“FIH”) Study

Compound A is being investigated in an ongoing Phase 1/2, open label, dose escalation and cohort expansion study to evaluate the safety, tolerability, pharmacokinetics and anti-tumor activity of Compound A in combination with palbociclib (IBRANCE®) in patients with estrogen receptor positive/human epidermal growth factor receptor 2 negative (ER+/HER2−) locally advanced or metastatic breast cancer, who have received prior hormonal therapy and chemotherapy in the locally advanced/metastatic setting. An overview of the FIH Study is provided in Table 1 below.

The FIH Study will assess the safety and tolerability, and determine the MTD and/or RP2D, of Compound A, in combination with palbociclib, in patients with locally advanced or mBC. The study will also assess clinical activity of Compound A at the RP2Ds in combination with palbociclib. Additional evaluation will include single- and multiple-dose pharmacokinetics and biochemical activity.

Study Design: Part C of the FIH Study is a Phase 1b evaluating the combination of Compound A and palbociclib.

### Method of Administration

Compound A was orally administered in combination with palbociclib (Part C), in 28-day cycles, in doses shown in Table 1. Compound A was supplied as 10 mg, 50 mg, and 100 mg strength immediate release tablets. Tableting excipients were inert, compendial components commonly employed in oral formulations, including lactose monohydrate and sodium stearyl fumarate.

Palbociclib was administered orally once a day at 125 mg/day for 21 days followed by 7 days off treatment for each 28-day cycle according to product labeling and in compliance with its local prescribing information.

As of the data cut-off date of 6 Jun. 2022, 176 patients have been treated in the FIH Study (Part C, n=27) and evidence of preliminary clinical activity has been observed.

TABLE 1
Overview of Part C FIH Study

| | | | Compound A Treatment |
|---|---|---|---|
| Study Title | Study Design | Study Population | Groups Evaluated |
| A Phase 1/2, open | FIH, open-label, sequential | Patients with | Part C - Phase 1b Combination |
| label, dose escalation, | group, dose escalation | histologically or | with palbociclib: |
| and cohort expansion | 3 + 3 and cohort expansion | cytologically confirmed | Cpd A at 180^{a}, 200, 400, or |
| clinical trial to evaluate | consisting of: | ER+/HER2− metastatic, | 500 mg QD plus palbociclib: |
| the safety, tolerability, | Part C - Phase 1b | recurrent, or locally | orally at 125 mg QD for 21 |
| and pharmacokinetics | Combination of Cpd A + | advanced unresectable | days followed by 7 days off |
| of Cpd A alone and in | Palbociclib | breast cancer for which | treatment period |
| combination with | | standard curative | |
| palbociclib | | therapy is no longer | |
| (IBRANCE ®) in | | effective or does not | |
| patients with estrogen | | exist | |
| receptor positive/human | | Age: ≥18 years | |
| epidermal growth factor | | | |
| receptor 2 negative | | | |
| (ER+/HER2−) locally | | | |
| advanced or metastatic | | | |
| breast cancer, who have | | | |
| received prior hormonal | | | |
| therapy and chemotherapy | | | |
| in the locally advanced/ | | | |
| metastatic setting | | | |

^{a}Patients initially enrolled into this cohort received 180 mg QD, but the dose was rounded up to 200 mg QD upon availability of the 100 mg tablets.

### The Clinical Pharmacology Study

Compound A is being investigated in an ongoing Phase 1 clinical pharmacology study to evaluate the effect of food or a PPI (esomeprazole) on the single-dose PK and safety of Compound A in healthy postmenopausal female volunteers (“participants”). An overview of the Clinical Pharmacology Study is provided in Table 2 below.

Study Design: The Clinical Pharmacology Study consists of 3 independent cohorts: 1) an open-label, randomized, 2-period, crossover Fed/Fasted cohort to determine food effects; 2) an open-label, 2-period, fixed sequence PPI cohort to evaluate interactions with esomeprazole; and 3) an open-label, randomized, 2 period, crossover rBA cohort to evaluate 2 tablet formulations.

As of the data cut-off date of 16 Jun. 2022, 47 participants have been treated in the 3 cohorts of Clinical Pharmacology Study. (14 participants in Fed/Fasted, 17 participants in PPI, and 16 participants in rBA cohorts). Each participant received 2 doses of 200 mg Compound A, 1 in each of the 2 periods, and the Compound A treatments were separated by a washout period of at least 14 days.

A dose of 200 mg Compound A has been well-tolerated in the participants, and no TRAEs of Grade 2 or higher were reported.

TABLE 2
Overview of the Clinical Pharmacology Study

| | | Study | |
|---|---|---|---|
| Study Title | Study Design | Population | Compound A Treatment Groups Evaluated |
| A Phase 1, | Open-label study | Healthy post- | Each dose cohort receives 200 mg Cpd A |
| multi-part, | with 3 independent | menopausal | following a 10-hour overnight fast. |
| open-label study | parts: | women with | |
| to evaluate the | Fasted/Fed: | BMI 18.5 to | Fed/Fasted Cohort: Cpd A dosing on Day 1 |
| effect of food or | 2-period, crossover | 35.0 kg/m² | and Day 15: |
| a proton-pump | Fed/Fasted cohort | and body | Fasted Period: fasting continues for at least |
| inhibitor | to determine food | weight 50 to | 4 hours after dosing |
| (esomeprazole) | effects | 120 kg, | Fed Period: Cpd A administered 30 min |
| and to evaluate | PPI: | inclusive. | after starting to eat a high-fat meal, then |
| the relative | 2-period, fixed | Age: 18 to | fasting for at least 4 hours after Cpd A |
| bioavailability of | sequence PPI | 70 years | administration |
| different tablet | cohort to evaluate | | PPI Cohort: |
| formulations on | interactions with | | Single-dose period: Cpd A administered in a |
| the single-dose | esomeprazole | | fed state^{c} on Day 1 and Day 19 |
| pharmacokinetics | rBA: | | Sequential-dose period: esomeprazole |
| and safety of Cpd | randomized, 2- | | delayed-release capsule (40 mg QD) at least |
| A in healthy | period, crossover | | 1 hour prior to food for 5 days from Days |
| post-menopausal | rBA cohort to | | 15-19. On Day 19 at least 90 min after |
| female volunteers | evaluate 2 tablet | | esomeprazole, Cpd A is administered in a |
| | formulations | | fed state^{d} |
| | | | rBA Cohort: |
| | | | 200 mg Cpd A administered as either 20% |
| | | | (reference) or 42% (test) drug load on Day 1 |
| | | | and Day 15 in a fed state^{c} |

^{c}Study drug administered 30 minutes after starting to eat a moderate-fat meal, then fasting for at least 4 hours after Compound A administration.

### Example 1: Pharmacokinetics (PK) in Patients with Breast Cancer (the FIH Study)

Preliminary PK data from Part C of the FIH Study (Compound A administered with palbociclib) were available from dose levels ranging from 180 to 500 mg QD. Preliminary results indicated dose-dependent increases in Cₘₐₓ and AUCₜₐᵤ for Compound A, Compound B, and the sum of Compound A+Compound B up to 500 mg QD on both Cycle 1 Day 1 and Day 15. The clinical DDI potential between Compound A and palbociclib was evaluated by comparing the plasma PK exposure parameters of palbociclib and Compound A (Cₘₐₓ and AUCₜₐᵤ) obtained in Part C on Cycle 1 Day 15 and the relevant PK data observed in the previously completed studies where palbociclib or Compound A was administered as a single agent. PK data from Part C suggested a lack of the effect of palbociclib on Compound A exposure, as evidenced by similar Cₘₐₓ and AUCₜₐᵤ of Compound A in patients receiving Compound A with palbociclib compared with those patients receiving Compound A monotherapy. On the other hand, preliminary Cₘₐₓ and AUCₜₐᵤ of palbociclib on Cycle 1 Day 15 after 125 mg QD palbociclib dosing in combination with 500 mg QD Compound A were approximately 18% to 25% and 34%, respectively, higher than those values observed in studies PALOMA-1 and PALOMA-2 when 125 mg QD palbociclib was administered (Pfizer. IBRANCE® (palbociclib). Product Monograph Including Patient Medication Information. Kirkland, Quebec: Pfizer Canada ULC; Revised: 15 Jul. 2021. Available at: www.pfizer.ca/sites/default/files/202107/Ibrance_PM_EN_243405_15 Jul. 2021.pdf; Durairaj C, Ruiz-Garcia A, Gauthier E R, et al. Palbociclib has no clinically relevant effect on the QTc interval in patients with advanced breast cancer. Anticancer Drugs. 2018 March; 29 (3): 271280). Further evaluation on DDI potentials between palbociclib and Compound A is ongoing.

### Example 2: Pharmacokinetics (PK) in Healthy Volunteers (the Clinical Pharmacology Study)

In the Clinical Pharmacology Study, preliminary PK parameters following a single 200 mg Compound A dose from all 3 cohorts were calculated. The median Tₘₐₓ ranged from 6.0 to 8.0 hours across the cohorts. The geometric mean T12 following a single 200 mg dose was approximately 40 hours under fed condition.

Statistical analysis showed that food intake significantly increased Compound A Cₘₐₓ and AUC_{inf} 3- to 2-fold, respectively, as compared with fasted conditions. Therefore, patients should be instructed to take Compound A with food. Compound A AUC values were similar when administered with PPI or without PPI, although PPI decreased Compound A median Cₘₐₓ about 18% which is not considered clinical meaningful. This indicates that there are no effects of PPI on exposure of Compound A when administered with a moderate-fat meal.

### Example 3: Safety in Healthy Volunteers (the Clinical Pharmacology Study)

Adverse events (AEs) were coded to system organ class and preferred term according to the medical dictionary for regulatory activities (MedDRA), version 24.1. The severity of adverse events was graded according to the National Cancer Institute Common Terminology Criteria for Adverse Events (NCI CTCAE) version 5.0.

A treatment-emergent adverse event (TEAE) is an AE occurring after the first dose of Compound A and within 30 days of the last Compound A dose regardless of study drug attribution or grade.

A treatment-related adverse event (TRAE) is an AE assessed as “Possibly Related,” “Probably Related,” or “Related” to Compound A by the investigator.

### Treatment-Emergent Adverse Events (TEAEs)

Regardless of study drug attribution and grade, TEAEs were observed in 15 of the 47 participants treated with 200 mg Compound A in the ongoing Clinical Pharmacology Study (all cohorts).

Fast/Fed Cohort (n=14): The most common TEAE (regardless of attribution to study drug) observed in ≥10% of participants was ligament sprain (2 participants, 14.3%). All other TEAEs were reported in <10% of participants.

All participants reporting a TEAE in the Fast/Fed Cohort experienced Grade 1 (21.4%) or Grade 2 (14.3%) TEAEs. No participants reported Grade 3 or higher TEAEs.

There were no clinically significant differences in TEAEs reported among the participants treated with Compound A in the fasted and fed conditions.

PPI Cohort (n=17): The most common TEAEs (regardless of attribution to study drug) observed in ≥10% of participants were headache (5 participants, 29.4%) and COVID-19 (2 participants, 11.8%).

All participants reporting a TEAE in the PPI Cohort experienced Grade 1 (47.1%) or Grade 2 (5.9%) TEAEs. No participants reported Grade 3 or higher TEAEs.

There were no clinically significant differences in TEAEs reported among the participants treated with Compound A with PPI and participants treated with Compound A without PPI. rBA Cohort (n=16): Only 1 participant (6.3%) reported a TEAE (dizziness, Grade 1).

There were no clinically significant differences in TEAEs reported among the participants treated with the 20% drug load and participants treated with the 42% drug load.

### Treatment Related Adverse Events (TRAEs)

A total of 3 of the total 47 participants had at least 1 TEAE considered potentially related to Compound A (regardless of severity grade).
Fast/Fed Cohort (n=14): 1 participant (7.1%) reported a TRAE of pollakiuria.
PPI Cohort (n=17): 2 participants (11.8%) reported at least 1 TRAE. TRAEs reported include nausea, headache, and pruritus (1 participant [5.9%]each).
rBA Cohort (n=16): No participants reported a TRAE.

Serious Adverse Events (SAEs), Serious Adverse Drug Reactions (SARs) and Deaths No SAEs, SARs or deaths were reported in the Clinical Pharmacology Study (all cohorts).

### Treatment-Emergent Adverse Events (TEAEs) Leading to Discontinuation

One healthy participant reported a TEAE that led to discontinuation of the Clinical Pharmacology Study. This participant was in the PPI cohort, treated with 200 mg Compound A and no PPI, and was unable to be dosed with Compound A for the second time, with PPI, due to COVID.

### Example 4: Safety and Efficacy in Patients with Breast Cancer (the FIH Study)

I. Safety in Patients with Breast Cancer (the FIH Study)

TEAEs and TRAEs from the clinical study database were coded to system organ class and preferred term using MedDRA version 22.0 and SAEs from the centralized safety database were coded using MedDRA version 25.0. The severity of adverse events was graded according to the NCI CTCAE version 5.0.

A TEAE is an AE occurring after the first dose of Compound A and within 30 days of the last Compound A dose regardless of study drug attribution or grade.

A TRAE is an AE assessed as “Possibly Related,” “Probably Related,” or “Related” to Compound A by the investigator.

Preliminary safety data for the FIH Study is presented below. Part C continues to enroll.

### Adverse Event Summary

Based on a 6 Jun. 2022 data cut-off, regardless of study drug attribution and grade, TEAEs were observed in 157 of the total 176 patients treated with Compound A in the ongoing FIH Study (all parts).

### Compound A Combination Therapies-Part C

Evaluation of preliminary safety data from patients treated with the combination of Compound A and palbociclib show TEAEs consistent with TEAEs observed after treatment with Compound A and palbociclib alone (Pfizer. IBRANCE® (palbociclib). US FDA Package Insert. Capsules, for oral use: Full Prescribing Information. New York, NY: Pfizer Laboratories; Revised: 04/2019. Available at: www.accessdata.fda.gov/drugsatfda_docs/label/2019/207103s0081bl.pdf).

Based on a 6 Jun. 2022 data cut-off, a total of 24 of 27 patients (88.9%) reported at least 1 TEAE in Part C.

The most common TEAEs (regardless of attribution to study drug) observed in ≥20% of patients, include neutrophil count decreased (51.9%), fatigue (44.4%), neutropenia (37.0%), platelet count decreased (33.3%), anemia (29.6%), nausea (25.9%), cough (22.2%), diarrhea (22.2%), and white blood cell decreased (22.2%).

### Compound a Combination Therapies-Part C-Combination of Compound A+Palbociclib:

Based on a 6 Jun. 2022 data cut-off, 5 of the 27 patients reported a total of 7 SAEs. All SAEs reported in Part C were assessed by the investigator as not related to Compound A. One SAE of Grade 3 bacteremia was assessed by the investigator as possibly related to palbociclib treatment.

II. Efficacy in Patients with Breast Cancer (the FIH Study)

Efficacy data collection is ongoing for Part C.

### Conclusion

Without wishing to be bound by theory, based on the foregoing Examples, a 200 mg daily dose may be beneficial for Compound A in combination with administration of a CDK4/6 inhibitor.

### Example 5: TACTIVE-U: Phase 1b/2 Umbrella Study of Compound a, a PROteolysis Targeting Chimera (PROTAC) Estrogen Receptor (ER) Degrader, Combined with Other Anticancer Treatments in ER+ Advanced or Metastatic Breast Cancer

Patients eligible for sub-studies A and B are aged ≥18 years, have histologically or cytologically confirmed ER+/HER2-advanced or metastatic breast cancer not amenable to surgical resection with ≥1 measurable lesion, and have received ≤2 lines of prior therapy for advanced or metastatic disease, including 1 line of any CDK4/6 inhibitor-based regimen. In sub-study A, patients will receive Compound A orally once daily (QD) in a dose escalation/de-escalation approach and abemaciclib orally twice daily (BID) continuously. In sub-study B, patients will receive Compound A orally QD in a dose escalation/de-escalation approach and ribociclib orally QD; Compound A will be given continuously and ribociclib will be given for 21 days followed by 7 days off treatment. For both sub-studies, the primary endpoint of the phase 1b portion is dose-limiting toxicities to determine the recommended phase 2 dose of Compound A in combination with abemaciclib or ribociclib. Secondary endpoints of phase 1b are progression-free survival (PFS), antitumor activity (overall response rate [ORR], clinical benefit rate [CBR], and duration of response [DOR]), safety (type, frequency, and severity of adverse events and laboratory abnormalities), and plasma concentrations of study drugs; AUCₜₐᵤ and Cₘₐₓ of Compound A+/−ribociclib will be determined for sub-study B. Phase 2 further evaluates the antitumor activity of the combinations; the primary endpoint is ORR and secondary endpoints include overall survival, PFS, antitumor activity (CBR and DOR), safety, plasma concentration of study drugs, and changes in circulating tumor DNA.

As of the data cutoff date 6 Jun. 2023, 4 patients have received Compound A 200 mg QD and abemaciclib 150 mg BID in sub-study A and 2 patients received Compound A 100 mg QD and ribociclib 600 mg QD in sub-study B.

### Example 6: Enhanced Efficacy of Compound A in Combination with CDK4/6 Inhibitors in ER+ Breast Cancer Models

### Methods

### Live-Cell Imaging Proliferation Assay

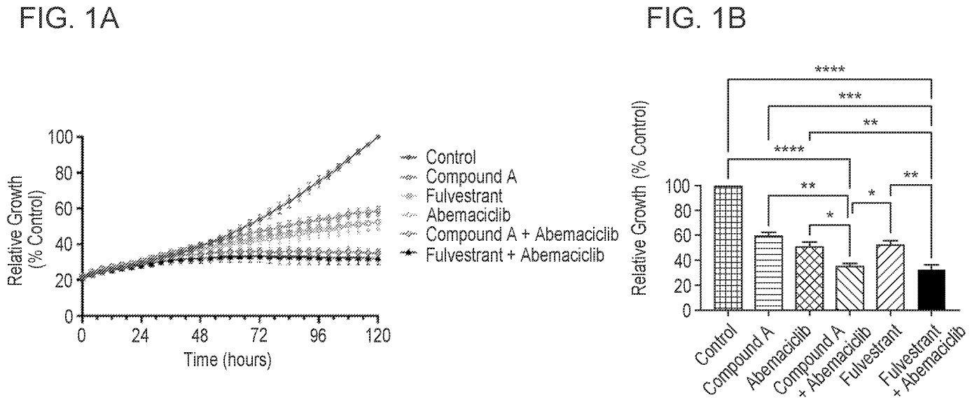

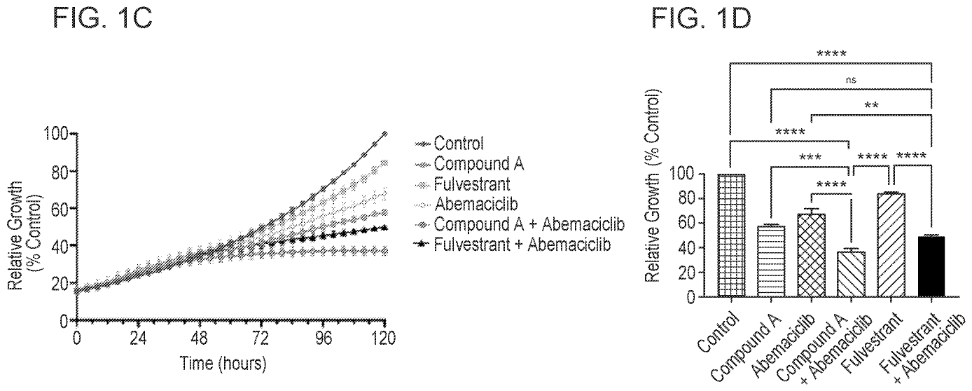

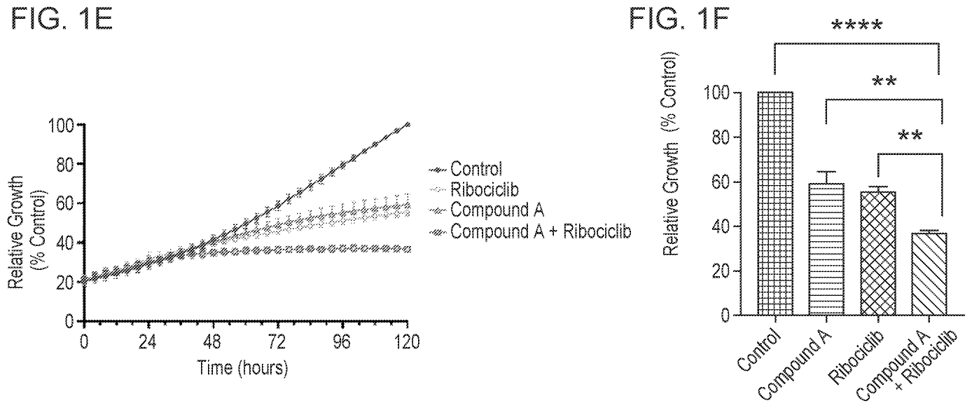

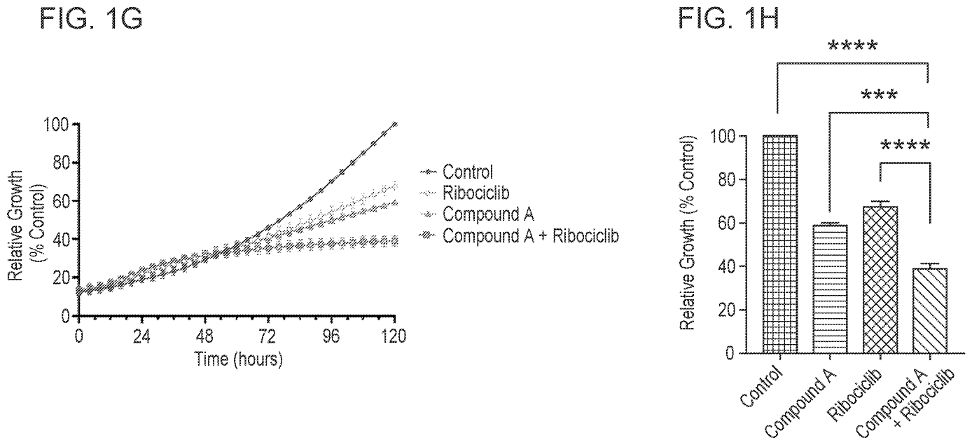

MCF7 or T47D cells were seeded in 6 well plates and treated with the indicated concentrations of compounds. The plate was then placed in the Incucyte® S3 Live-Cell Analysis System and images were acquired every 4 hours for a total of 5 days. Data were analyzed using the Incucyte® Software v2020C which quantified cell surface area coverage as confluence values. Relative growth was calculated for all timepoints for all growth conditions relative to the confluence value observed for the control at 120 hours (FIGS. 1A and 1C). Graphing and statistical analyses were performed using Graphpad Prism (GraphPad Software).

In MCF7 and T47D cells treated with Compound A or fulvestrant as single agents or in combination with abemaciclib at concentrations approximating their respective GIso for 5 days, mean decreases in MCF7 cell growth for Compound A, abemaciclib and the combination were 41%, 49% and 65%, respectively, relative to control cells (FIGS. 1A-1B). For fulvestrant and fulvestrant plus abemaciclib, mean decreases in MCF7 cell growth were 48% and 68%, respectively. Mean decreases in T47D cell growth for Compound A, abemaciclib and the combination were 42%, 32% and 63%, respectively, and for fulvestrant and fulvestrant plus abemaciclib, 16% and 50%, respectively (FIGS. 1C-1D). Treatment with single agent or combined Compound A and ribociclib yielded similar results. After 5 days of treatment at concentrations approximating their respective GIso, mean decreases in MCF7 cell growth for Compound A, ribociclib and the combination were 41%, 44% and 63%, respectively, relative to control cells (FIGS. 1E-1F); mean changes in T47D cell growth for Compound A, ribociclib and the combination were 41%, 32% and 61%, respectively (FIGS. 1G-1H). The combination of Compound A with either abemaciclib or ribociclib demonstrated significantly greater inhibition of growth of both MCF7 and T47D cells when compared to either single agent alone. These results were similar to those observed with fulvestrant when combined with either abemaciclib or ribociclib.

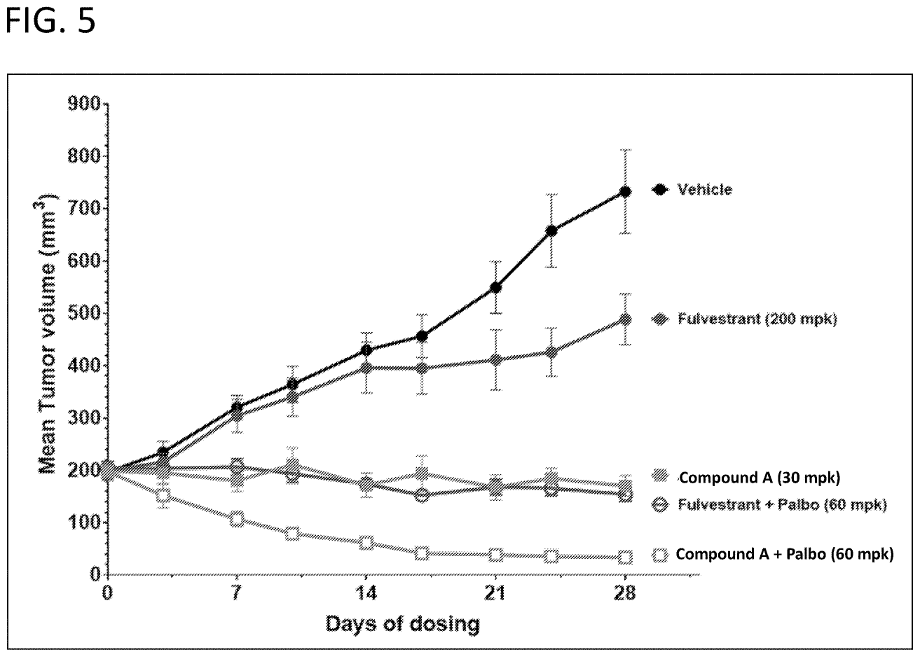

When compared to single agent Compound A activity (105% TGI) in this model (FIG. 5), the combination of Compound A and palbociclib resulted in tumor regressions (131% TGI). In contrast, single-agent fulvestrant, which was dosed subcutaneously, resulted in only modest TGI (46% TGI), while the combination of fulvestrant and palbociclib resulted in improved inhibition of tumor growth (108% TGI) but not to the levels achieved with Compound A and palbociclib. Similar results were observed in the tamoxifen-resistant xenograft model, where the combination of Compound A and palbociclib caused greater TGI compared to either single agent alone.

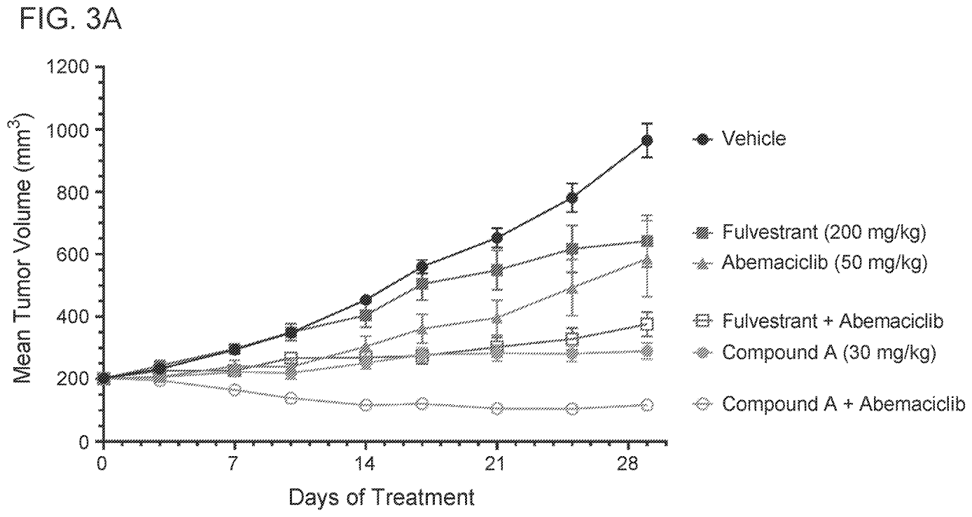

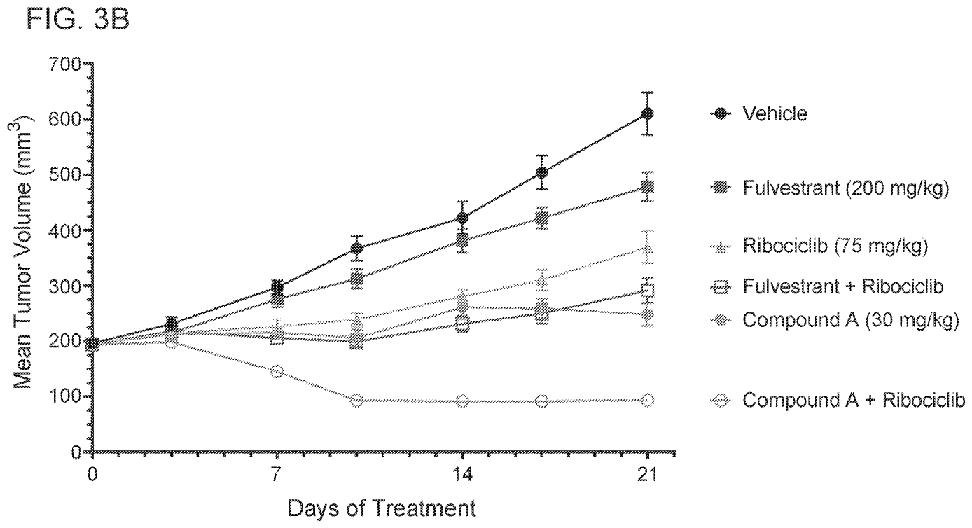

The in vivo effects of combining Compound A or fulvestrant with abemaciclib were also evaluated in MCF7-tumor bearing mice. As single agents, Compound A and abemaciclib treatment resulted in 88% and 50% TGI, respectively, whereas the combination resulted in greater growth inhibition (111% TGI) than either single agent alone (FIG. 3A). Fulvestrant as a single agent demonstrated modest TGI (42%) and enhanced TGI (77%) when combined with abemaciclib. However, TGI observed with the combination of fulvestrant and abemaciclib did not achieve the level observed with the combination of Compound A and abemaciclib.

Treatment of MCF7 tumor-bearing mice with Compound A and ribociclib as single agents resulted in 87% TGI and 58% TGI, respectively, whereas the combination resulted in greater TGI (124%) than either single agent alone (FIG. 3B). As a single agent fulvestrant demonstrated modest TGI (31%) and enhanced TGI (76%) in combination with ribociclib but not to the levels achieved with the combination of Compound A and ribociclib, similar to observations with the combination of fulvestrant and abemaciclib.

Taken together, these results demonstrate that, in BC tumor models, Compound A displays anti-tumor activity as a single agent and greater anti-tumor activity in combination with the CDK4/6 inhibitors abemaciclib and ribociclib and exhibits greater anti-tumor effects than those observed with the combination of fulvestrant with either of these CDK4/6 inhibitors.

### Dose-Response Matrix Assay

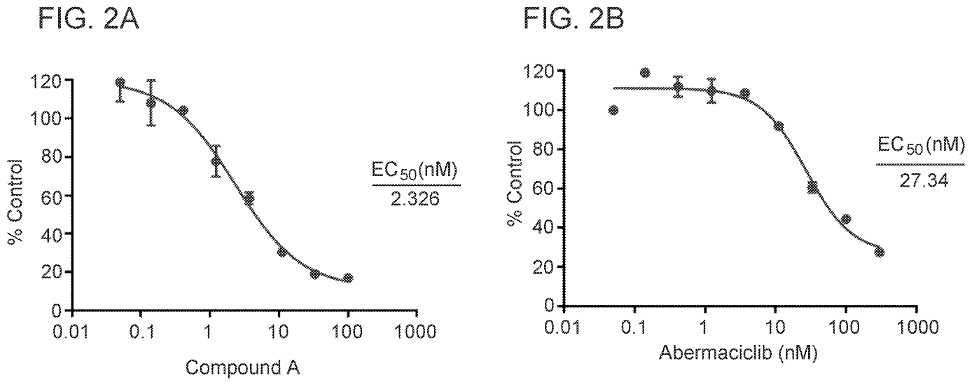

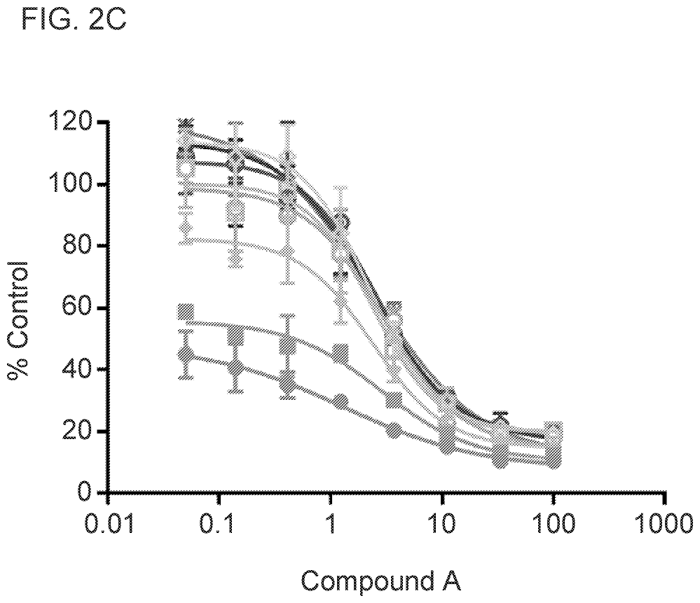

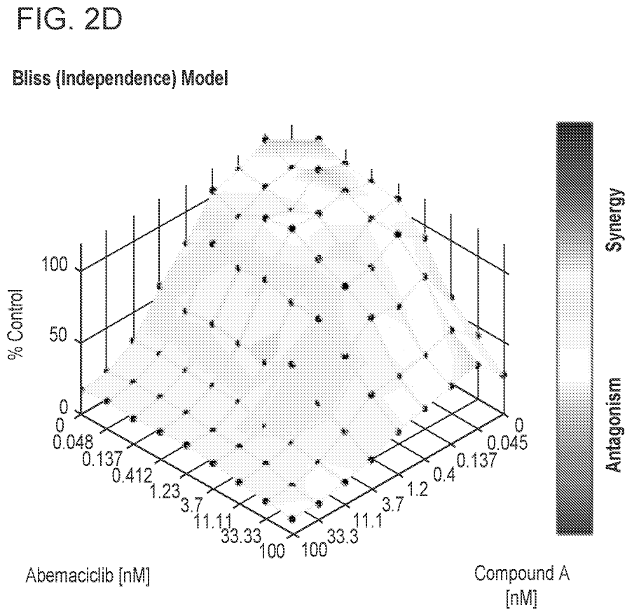

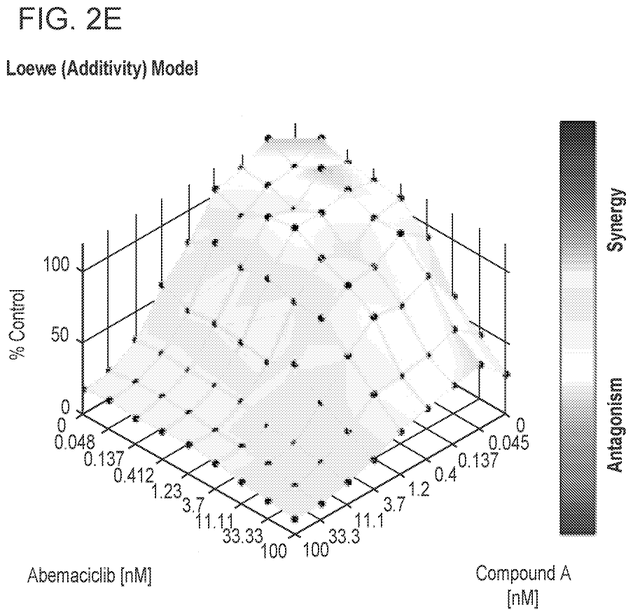

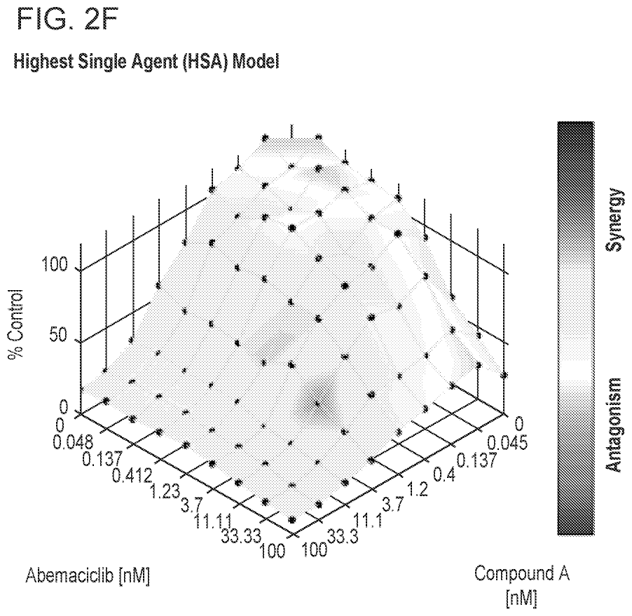

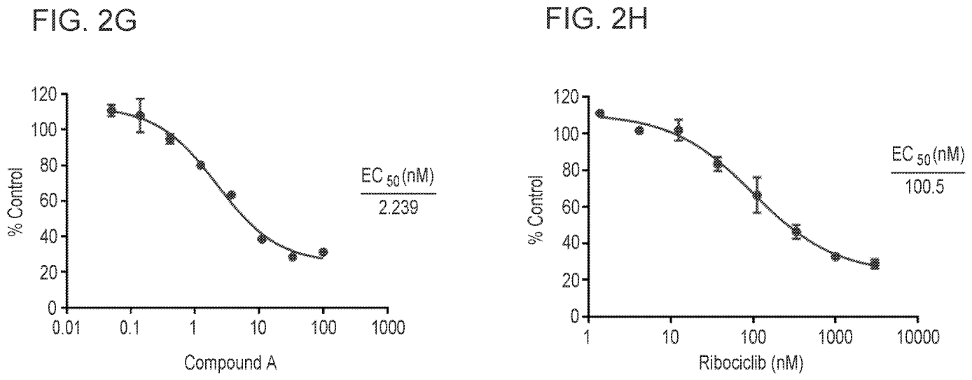

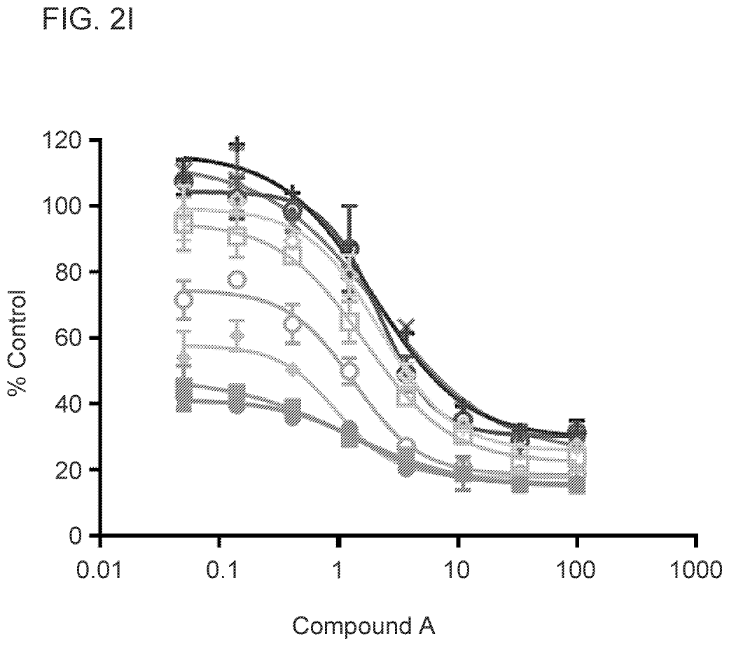

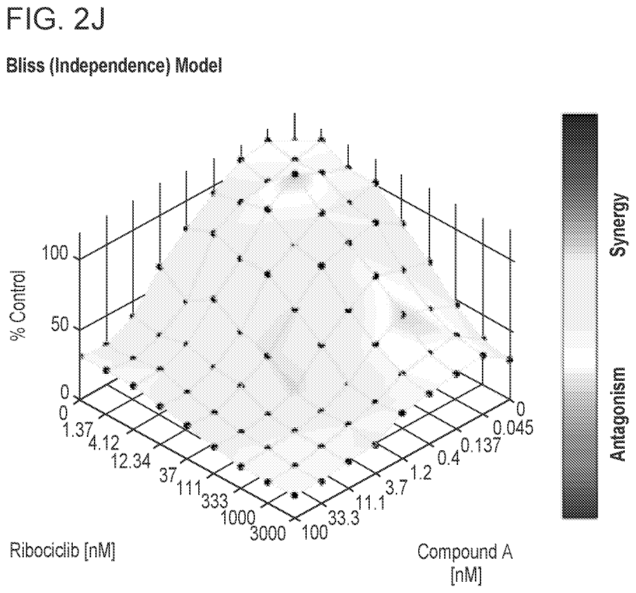

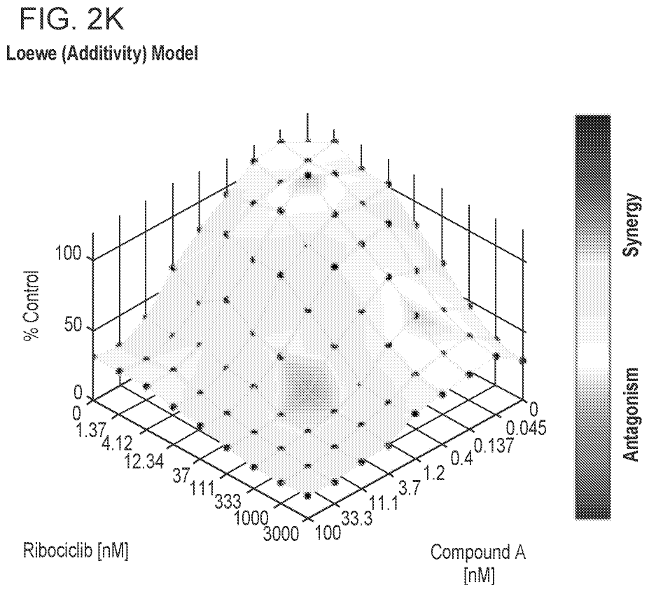

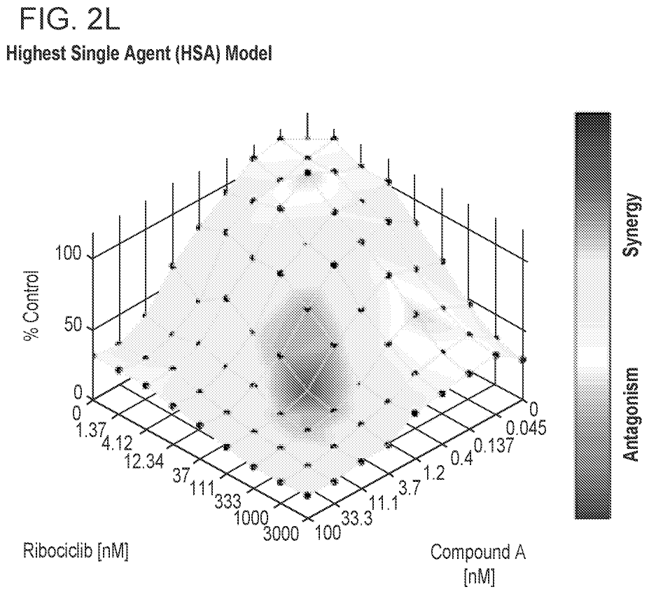

Cells were seeded at 2×10³ cells in 200 μl of media per well in 96 well plates and incubated overnight at 37° C. Compound A and Abemaciclib concentrations curves were started at 100 nM for an 8-point concentration curve ranging from 100 to 0.046 nM (FIGS. 2A, 2C, and B). Ribociclib concentration curves were started at 3000 nM for an 8-point concentration curve ranging from 3000 to 1.37 nM (FIG. 2B). At Day 5 cell viability was measured using Cell-Titer Glo and CTG data were analyzed with the Combenefit Software (Veroli G Y D, Fornari C, Wang D, Mollard S, Bramhall J L, Richards F M, et al. Combenefit: an interactive platform for the analysis and visualization of drug combinations. Bioinformatics. 2016; 32:2866-8). The combination of either abemaciclib or ribociclib with Compound A demonstrated enhanced cell growth inhibition compared to single agent treatments and resulted in synergism by Bliss, Loewe and HSA models, with the exception of the Bliss model for T47D cells.

### MCF7 Xenograft Model

Briefly, MCF7 cells were orthotopically implanted into the mammary fat pads of NOD/SCID female mice. 17β-estradiol 0.72 mg 90-day pellet (Innovative Research of America) were implanted 2-3 days prior to MCF7 cell implant. For combination arms, Compound A was administered first followed by combination partners 1 hour later. Compound A- and/or combination partner-treated mice were dosed orally once daily. Fulvestrant-treated mice were dosed subcutaneously twice per week for 2 weeks followed by once weekly for 2 weeks.

More specifically, synergism between Compound A and abemaciclib and ribociclib was followed up in-vivo using the MCF7 orthotopic xenograft model. Eight- to ten-week-old female NOD/SCID mice were surgically implanted with a 0.36 mg 90-day release 17β-estradiol pellet subcutaneously. One to two days later each mouse was injected with 5×10⁶/200 μL MCF7 cells into one mammary fat pad. Cells were prepared in a 50/50 RPMI-1640 phenol red-free media/Corning Matrigel Membrane Matrix mix at 25×10⁶ cells/ml. Dosing initiated once tumors reached an average of 200 mm³. Where oral combinations were dosed, Compound A (30 mg/kg) was dosed first and abemaciclib (30 mg/kg) and ribociclib (75 mg/kg) 30-60 minutes later. Fulvestrant (200 mg/kg, subcutaneously; twice per week for 2 weeks followed by once-weekly for 2 weeks) was also evaluated as a single agent and in combination with abemaciclib and ribociclib. Drugs indicated were dosed as single agents or in combination to 10 mice/arm.

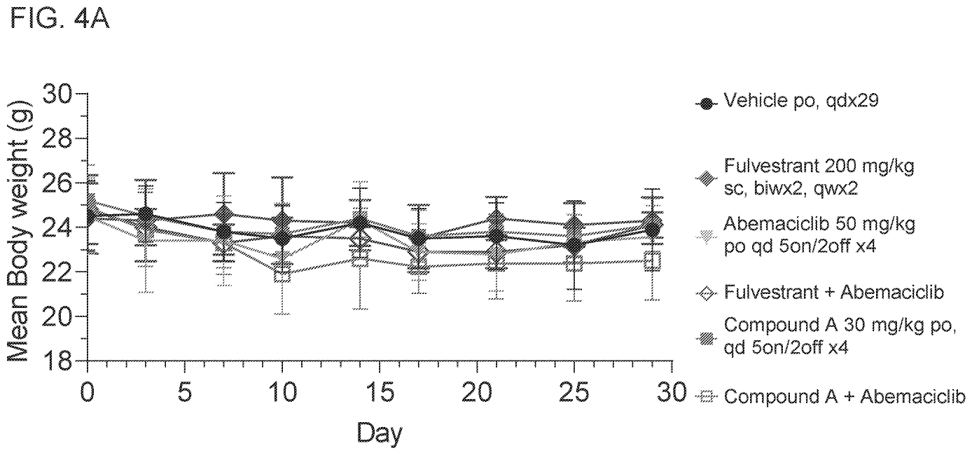

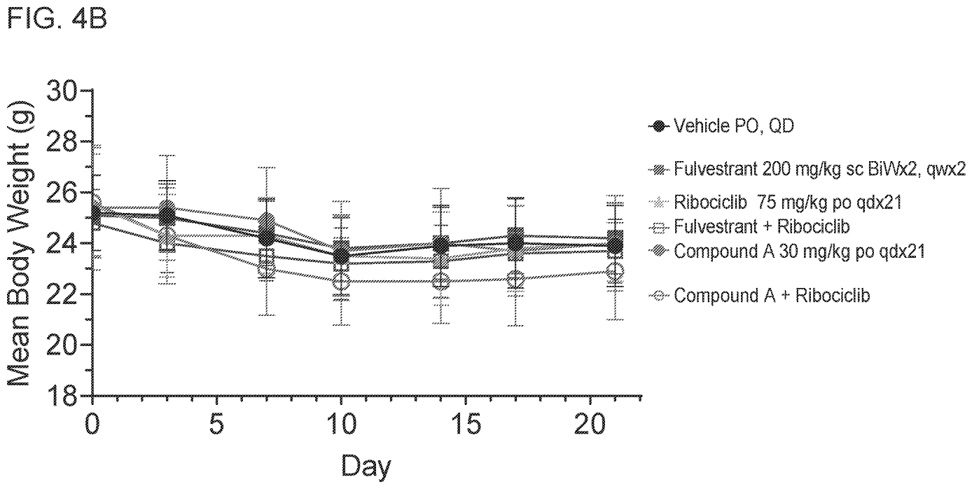

Compound A and abemaciclib and ribociclib were dosed at 5 ml/kg volume once daily for 28 days (qdx28). Vehicles for Compound A was 2% Tween 80/PEG400. Vehicle for abemaciclib was 1% hydroxymethyl cellulose (HEC) in 20 mM HCl pH2. Vehicle for ribociclib was 0.5% methylcellulose. Vehicle for fulvestrant was 10% w/v ethanol, 10% w/v benzyl alcohol, 15% w/v benzyl benzoate, made up to 100% w/v with castor oil. A single-day dosing holiday) was implemented on all arms if any body weight loss approached 10% (days 11, 12, 19, and 20 of the ribociclib study). Body weights were well maintained with a single day dosing holiday (FIGS. 4A and 4B). Tumor volumes were measured twice per week in efficacy studies and calculated using (width²×length)/2, where all measurements are in millimeters (mm) and the tumor volume is in mm³. Body weights were recorded twice per week. At study termination mice were euthanized 18-hours post-last dose ad harvested tissue was snap-frozen on dry ice. Tumor growth inhibition (TGI) was calculated as follows, where tumor volume is in mm³:

$$TGI ( \% ) = [ 1 - \frac{\begin{matrix}( Tumor volume , compound , Day X ) - \\ ( Tumor volume , compound , Day 0 )\end{matrix}}{\begin{matrix}( Tumor volume , vehicle , Day X ) - \\ ( Tumor volume , vehicle , Day 0 )\end{matrix}} ] \times 100$$

At the end of the abemaciclib study, single-agent Compound A and abemaciclib inhibited tumor growth by 88% and 50%, respectively. The growth inhibition for Compound A in combination with abemaciclib was 111%. Fulvestrant inhibited tumor growth by 42% as a single agent and when combined with abemaciclib by 77%.

At the end of the ribociclib study, single-agent Compound A and ribociclib inhibited tumor growth by 87% and 58%, respectively. The growth inhibition for Compound A in combination with ribociclib was 124%. Fulvestrant inhibited tumor growth by 31% as a single agent and by 76% when combined with ribociclib.

Data are presented as mean±standard error of the mean.

### Findings

In vitro studies revealed evidence of synergistic interactions between Compound A and the CDK4/6 inhibitors abemaciclib and ribociclib. FIGS. 1 and 2 show Compound A in combination with CDK4/6 inhibitors (i.e., abemaciclib and ribociclib) demonstrates enhanced efficacy and evidence of synergy in vitro.

Compound A in combination with the CDK4/6 inhibitors abemaciclib and ribociclib led to enhanced tumor regressions in MCF7 xenografts as compared to single agents alone.

Compound A displayed greater anti-tumor activity in combination with abemaciclib or ribociclib than that observed with fulvestrant in combination with these agents.

Taken together, these data highlight the potential utility of Compound A as a combination partner for clinically relevant targeted agents for treatment of early and late-stage ER+ disease.

### Example 6: a Study to Learn about Compound 1 in People with ER+/HER2-Advanced Breast Cancer in China

### Brief Summary

The purpose of this clinical trial is to learn about the pharmacokinetics. safety and tolerability of compound 1 for the potential treatment of advanced estrogen receptor positive and human epidermal growth factor receptor 2 negative breast cancer.

This study is seeking participants have −ER+/HER2− advanced breast cancer
received at least 1 line of endocrine therapy with or without CDK4/6 inhibitor
received up to 2 prior regimens of chemotherapy for advanced setting.

All participants in this study will receive Compound A.

Compound A will be given by mouth at home once a day.

The experiences of people receiving the study medicine will be examined. This will help determine if the study medicine is safe and effective.

Participants will take part in this study until their cancer is no longer responding. During this time, they will have visits at the study clinic about every 4 weeks.

All participants will stay at the study clinic for 10 days and 9 nights.
Study type: Interventional
Study phase: Phase 1
Study arms: One study arm
Masking: No masking
Enrollment number: 9

### Interventions

| | | |
|---|---|---|
| Arm title | Type | Description |

| | |
|---|---|
| Rosuvastatin with/without Compound A | Experimental |

| | | | | |
|---|---|---|---|---|
| Intervention | | Associated | | Other |
| Name | Type | Arms | Description | Names |

| | | | |
|---|---|---|---|
| Compound A | Drug | Compound A | Compound A will be administered |
| | | | orally once daily with food (e.g., a |
| | | | light meal of approximately 400 to |
| | | | 600 calories which includes a |
| | | | mixture of fat carbohydrates, and |
| | | | protein) at RP3D for monotherapy |
| | | | defined in study ARV-471-mBC- |
| | | | 101, in continuous dosing over 28- |
| | | | day cycles |

### Outcome Measures

| Outcome Measure | Timeframe | Description |
|---|---|---|
| Single dose Cmax (Maximum | 0, 1, 2, 3, 4, 6, 8, 12, 24, 48, | Maximum plasma |
| plasma concentration) | 72, 96 hours post-dose Up to | concentration |
| | Day 71 | |
| Single dose AUCtau | 0, 1, 2, 3, 4, 6, 8, 12 hours | Area under the concentration- |
| | post-dose Up to Day 71 | time profile from time zero to |
| | | time tau (τ), the dosing |
| | | interval, where tau = 24 hours |
| | | (QD dosing) |
| Multiple dose Cmax | 0, 1, 2, 3, 4, 6, 8, 12, 24, 48, | Maximum Observed Plasma |
| | 72, 96 hours post-dose | Concentration (Cmax) |
| Multiple dose AUCtau | 0, 1, 2, 3, 4, 6, 8, 12 hours | Area under the concentration |
| | post-dose | curve from time 0 to end of |
| | | dosing interval (AUCtau), |
| | | where dosing interval was 12 |
| | | hours. |

### Secondary Outcome Measures

| Outcome Measure | Timeframe | Description |
|---|---|---|
| Objective | Baseline up | Percentage of participants with objective response based |
| Response Rate - | to 24 weeks | assessment of confirmed complete response (CR) or |
| Percentage of | | confirmed partial response (PR) according to Response |
| Participants With | | Evaluation Criteria in Solid Tumors (RECIST). |
| Objective | | Confirmed responses are those that persist on repeat |
| Response | | imaging study at least 4 weeks after initial |
| | | documentation of response. |
| | | Per RECIST v1.1: CR was defined as complete |
| | | disappearance of all target lesions and non-target |
| | | disease, with the exception of nodal disease. All nodes, |
| | | both target and non-target, must decrease to normal |
| | | (short axis <10 mm). No new lesions. PR was defined |
| | | as >=30% decrease under baseline of Percentage of |
| | | participants with objective response based assessment of |
| | | complete response (CR) or partial response (PR) |
| | | according to Response Evaluation Criteria in Solid |
| | | Tumors (RECIST). |
| Percentage of | Baseline up | Percent of participants with confirmed complete |
| Participants With | to 24 weeks | response (CR), partial response (PR) or stable disease |
| Clinical Benefit | | (SD) for at least 24 weeks on study according to |
| | | RECIST. |
| | | Per RECIST v1.1: CR was defined as complete |
| | | disappearance of all target lesions and non-target |
| | | disease, with the exception of nodal disease. All nodes, |
| | | both target and non-target, must decrease to normal |
| | | (short axis <10 mm). No new lesions. PR was defined |
| | | as >=30% decrease under baseline of the sum of diameters |
| | | of all target lesions. The short axis was used in the sum |
| | | for target nodes, while the longest diameter was used in |
| | | the sum for all other target lesions. No unequivocal |
| | | progression of non-target disease. No new lesions. SD |
| | | was defined as not qualifying for CR, PR, Progressive |
| | | Disease (PD). |
| Duration of | From the | DOR: time from date of first documented response (CR |
| Objective | date of first | or PR) to first documented progression or death due to |
| Response (DOR) | documented | underlying cancer. RECIST V1.1, a) CR: disappearance |
| | response (CR | of all lesions; any pathological lymph nodes (TLs) or |
| | or PR) to the | non-pathological (non-TLs) must have reduction in |
| | first | short axis to <10 mm; normalization of tumor marker |
| | documented | level for non-TLs; b) PR: >=30% decrease in sum of |
| | progression | diameter (dia) of all TLs, referring baseline sum of dia; |
| | or death | c) PD: >=20% increase in sum of diameter of all |
| | | measured target lesions taking as reference smallest sum |
| | | of diameter of all target lesions recorded at or after |
| | | baseline, sum must also be absolute increase of >=5 mm. |
| | | Unequivocal progression of existing non-target |
| | | lesions. Appearance of at least 1 new lesion. If a |
| | | participant with a CR or PR had no PD or death due to |
| | | underlying cancer, participants was censored at date of |
| | | last adequate tumor assessment. |
| Presence (rate) or | Baseline up | To identify biomarkers (ESRI mutation) of complete |
| absence of blood | to end of | response and progression if occurs |
| biomarkers | treatment | |
| Number of | From | Criteria for notable ECG values were as follow: QT |
| Participants With | baseline up | interval (in millisecond [msec]) new (newly occurring |
| Notable | to 28 days | post-baseline value) greater than (>) 450, 480, 500, |
| Electrocardiogram | after last | increase from baseline >30, |
| (ECG) Values | dose of study | increase from baseline >60; corrected QT interval by |
| | drug | Fredericia formula (QTcF) in msec new (newly |
| | | occurring post-baseline value) >450, 480, 500, increase |
| | | from baseline >30, increase from baseline >60; |
| | | corrected QT interval by Bazett's formula (QTcB) in |
| | | msec new (newly occurring post-baseline value) >450, |
| | | 480, 500, increase from baseline >30, increase from |
| | | baseline >60; heart rate in bpm new (newly occurring |
| | | post-baseline value) <60 and >100. |
| Number of | Baseline | Hemoglobin (HGB), hematocrit, erythrocytes |
| Participants With | (Day 1) up to | (ery.), HDL cholesterol (chl.) <0.8*lower limit of |
| Laboratory | at least 28 | normal(LLN); reticulocytes (ret.), ret./ery. |
| Abnormalities | days after | (%) <0.5*LLN, >1.5*upper limit of normal (ULN); ery. |
| | last dose of | mean corpuscular (EMC) volume, EMC HGB, EMC HGB |
| | study drug | |
| Number of | Baseline up | Vital signs (temperature, respiratory rate, pulse, systolic |
| Participants With | to 28 days | and diastolic blood pressure) were obtained with |
| Clinically | after last | participant in the seated position, after having sat calmly |
| Significant Change | dose of study | for at least 5 minutes. Clinical significance of vital signs |
| From Baseline in | drug | was determined at the investigator's discretion. |
| Vital Signs | | |
| Number of | Baseline up | Counts of participants who had treatment-emergent |
| Participants With | to 28 days | adverse events (TEAEs), defined as newly occurring or |
| Adverse Events | after the last | worsening after first dose. Relatedness to [study drug] |
| (AEs) by type, | dose of study | was assessed by the investigator. Participants with |
| frequency, severity | drug | multiple occurrences of an AE within a category were |
| (as graded by NCI | | counted once within the category. An adverse event |
| CTCAE version | | (AE) was any untoward medical occurrence in a |
| 5.0), timing, | | participant who received study drug without regard to |
| seriousness and | | possibility of causal relationship. As per CTCAE |
| relationship to | | version 4, Grade 1 = mild AE; Grade 2 = moderate AE; |
| study treatment | | Grade 3 = severe AE; Grade 4 = life-threatening or |
| | | disabling AE; Grade 5 = death related to an AE. |
| Single dose Tₘₐₓ | 0, 1, 2, 3, 4, | Time at which Cmax occurred |
| | 6, 8, 12, 24, | |
| | 48, 72, 96 | |
| | hours post- | |
| | dose | |
| single dose AUCₗₐₛₜ | up to the last | Area under the concentration-time profile from time |
| | quantifiable | zero to the time of the last quantifiable concentration |
| | concentration | (Clast) |
| Single dose | Up to Day 71 | Compound 2 to compound 1 ratio for Cmax |
| MRCₘₐₓ | | |
| Single dose | 0 (pre-dose), | Area under the concentration-time profile from time |
| AUCinf | 0.5, 1, 1.5, 2, | zero extrapolated to infinite time |
| | 3, 4, 6, 8, 12, | |
| | 24, 48, and | |
| | 72 hours | |
| | post-dose | |
| Single dose CL/F | Week X | Clearance of a drug is a measure of the rate at which a |
| | | drug is metabolized or eliminated by normal biological |
| | | processes. Clearance obtained after oral dose (apparent |
| | | oral clearance) is influenced by the fraction of the dose |
| | | absorbed. Clearance was estimated from population |
| | | pharmacokinetic (PK) modeling. Drug clearance is a |
| | | quantitative measure of the rate at which a drug |
| | | substance is removed from the blood. |
| Single dose Vz/F | 0 (pre-dose), | Apparent volume of distribution |
| | 1, 2, 4, 5, 6, | |
| | 8, 10, 12, 24, | |
| | 36, 48, 72, | |
| | 96, and 144 | |
| | hrs. post | |
| | dose | |
| Single dose t½ | 0, 1, 2, 3, 4, | Terminal half-life |
| | 6, 8, 12, 24, | |
| | 48, 72, 96 | |
| | hours post- | |
| | dose | |
| Multiple dose | 0, 1, 2, 3, 4, | Time to Reach Maximum Observed Plasma |
| Tmax | 6, 8, 12, 24, | Concentration |
| | 48, 72, 96 | |
| | hours post- | |
| | dose | |
| Multiple dose | 0, 1, 2, 3, 4, | Time at which Cmax occurred |
| Tmax | 6, 8, 12, 24, | |
| | 48, 72, 96 | |
| | hours post- | |
| | dose | |
| Multiple dose | Up to Day 71 | Compound 2 to compound 1 ratio for Cmax |
| MRCmax | | |
| Rac | Up to day 71 | Accumulation ratio based on AUC (observed) |
| t½eff | Up to day 71 | Effective half-life (t½eff) based on accumulation ratio |
| Multiple dose CL/F | Up to day 71 | Clearance of a drug is a measure of the rate at which a |
| | | drug is metabolized or eliminated by normal biological |
| | | processes. Clearance obtained after oral dose (apparent |
| | | oral clearance) is influenced by the fraction of the dose |
| | | absorbed. Clearance was estimated from population |
| | | pharmacokinetic (PK) modeling. Drug clearance is a |
| | | quantitative measure of the rate at which a drug |
| | | substance is removed from the blood. |
| Multiple dose t½ | 0, 1, 2, 3, 4, | Terminal half-life |
| | 6, 8, 12, 24, | |
| | 48, 72, 96 | |
| | hours post- | |
| | dose | |

### Eligibility

| Age | All |
|---|---|
| Age Limits | Min. Age: 18 |
| | Max. Age: N/A |
| Accepts Healthy Volunteers | No |
| Eligibility Criteria | Inclusion Criteria: - Histological or |
| | cytological diagnosis of breast cancer with |
| | evidence of ER+/HER2− locally advanced or |
| | metastatic disease, which is not amenable to |
| | surgical resection or radiation therapy with |
| | curative intent. - Received at least 1 line of |
| | SOC of endocrine therapy with or without |
| | CDK4/6 inhibitor for locally advanced or |
| | metastatic disease. - Up to 2 prior regimens of |
| | chemotherapy for advanced or metastatic |
| | disease setting are allowed. |
| | Exclusion Criteria: - Known active |
| | uncontrolled or symptomatic CNS metastases, |
| | carcinomatous meningitis, or leptomeningeal |
| | disease as indicated by clinical symptoms, |
| | cerebral edema, and/or progressive growth. |
| | Participants with a history of CNS metastases |
| | or cord compression are eligible if they have |
| | been definitively treated (e.g., radiotherapy, |
| | stereotactic surgery) and clinically stable |
| | (including patients with residual CNS |
| | symptoms/deficits) off enzyme-inducing |
| | anticonvulsants and steroids for at least 28 |
| | days prior to first dose of study |
| | drug. -Any of the following in the previous 6 |
| | months: myocardial infarction, |
| | severe/unstable angina, coronary/peripheral |
| | artery bypass graft, symptomatic congestive |
| | heart failure (New York Heart Association |
| | Class III or IV), cerebrovascular accident, |
| | transient ischemic attack, or symptomatic |
| | pulmonary embolism or other clinically |
| | significant episode of thromboembolic |
| | disease, congenital long QT syndrome, |
| | Torsade de Pointes, serious conduction |
| | system abnormalities (e.g., bifascicular block |
| | defined as right bundle branch and left |
| | anterior or posterior hemiblock, 3rd degree |
| | AV block), clinically important arrhythmias, |
| | left anterior hemiblock (bifascicular block), |
| | ongoing cardiac dysrhythmias of NCI |
| | CTCAE Grade ≥2, atrial fibrillation of any |
| | grade. |

### Example 6: Treatment-Emergent AEs (TEAEs)

As of 6 Jun. 2023, a total of 46 participants (100%) who received Compound A in combination with palbociclib 125 mg reported at least 1 TEAE.

The most common TEAEs observed in >20% of patients across all Compound A doses combined with palbociclib 125 mg, listed in decreasing order of frequency, were: neutropenia, fatigue, platelet count decreased, anemia, constipation, nausea, white blood cell count decreased, diarrhea and electrocardiogram QT prolonged (Table 4). Grade 3 and 4 TEAEs occurred in 23 (50%) and 19 (41.3%) of participants respectively. Neutropenia was the most common Grade 3 or Grade 4 TEAE and occurred in 47.8% (Grade 3) and 41.3% (Grade 4) of participants, respectively.

TABLE 3
TEAEs ≥5% by Preferred Term and Maximum CTCAE
Grade 3 and above - Study ARV-471-mbc-101 (Part C)

| | AE Preferred Term | Any | Grade | Grade | Grade |
|---|---|---|---|---|---|
| Dose Group | [n (%)] | Grade | 3 | 4 | 5 |
| Any treatment-emergent AE | | 46 (100.0) | 23 (50.0) | 19 (41.3) | 0 |
| Compound A All Dose Group + | Neutropenia | 46 (100.0) | 22 (47.8) | 19 (41.3) | 0 |
| Palbociclib 125 mg (TEAE) | | | | | |
| Compound A 200 mg + | Neutropenia | 21 (100.0) | 10 (47.6) | 8 (38.1) | 0 |
| Palbociclib 125 mg (TRAE) | | | | | |

* Neutropenia was categorized according to the MedDRA preferred terms neutropenia and neutrophil count decreased.
Note:
Treatment-emergent is defined as AE occurring on/after date of first study dose and within 30 days of last study dose.
Note:
For each Preferred Term, patients are counted only once.
Note:
Adverse Events were coded using MedDRA version 22.0.
Source: BDM TAE04C: TEAE by Preferred Term and Maximum CTCAE Grade (Part C) - All Dose Group All-Treated Analysis Set and BDM TAE04RELC2: Compound A or Palbociclib TRAE by Preferred Term and Maximum CTCAE Grade (Part C) - 200 mg All-Treated Analysis Set
Data cutoff date: 6 JUN. 2023

In patients treated with Compound A 200 mg and palbociclib 125 mg, the most common TEAEs observed in ≥20% of patients include neutropenia (100.0%) fatigue (66.7%), platelet count decreased (52.4%), anemia (42.9%), constipation (33.3%) alanine aminotransferase increased (23.8%) aspartate aminotransferase increased (23.8%) back pain (23.8%), dizziness (23.8%) and white blood cell count decreased (23.8%).

All patients experienced neutropenia in the Compound A 200 mg and palbociclib 125 mg cohort, of which 8 participants (38.1%) reported G4 neutropenia. Median time to first neutropenia episode of any grade was 14.5 days for patients treated with Compound A 200 mg in combination with palbociclib and is consistent with the IBRANCE USPI. All neutropenia episodes were reversible, noncumulative, and medically manageable with/without supportive care and/or dose reduction. Palbociclib dose reduction was reported in 13 participants (61.9%).

TABLE 4
Treatment-emergent Adverse Events ≥10% by Preferred
Term (Part C) -All-Treated Analysis Set (ARV-471-mBC-101)

| | 180 mg + | 200 mg + | 400 mg + | 500 mg + | |
|---|---|---|---|---|---|
| | Palbo | Palbo | Palbo | Palbo | Total |
| AE Preferred Term [n (%)] | (N = 2) | (N = 21) | (N = 3) | (N = 20) | (N = 46) |
| Any treatment-emergent adverse events | 2 (100) | 21 (100) | 3 (100) | 20 (100) | 46 (100) |
| Neutropenia* | 2 (100) | 21 (100) | 3 (100) | 20 (100) | 46 (100) |
| Fatigue | 2 (100) | 14 (66.7) | 1 (33.3) | 12 (60.0) | 29 (63.0) |
| Platelet count decreased | 0 | 11 (52.4) | 2 (66.7) | 10 (50.0) | 23 (50.0) |
| Anemia | 0 | 9 (42.9) | 2 (66.7) | 8 (40.0) | 19 (41.3) |
| Constipation | 0 | 7 (33.3) | 0 | 6 (30.0) | 13 (28.3) |
| Nausea | 1 (50.0) | 4 (19.0) | 1 (33.3) | 7 (35.0) | 13 (28.3) |
| White blood cell count decreased | 0 | 5 (23.8) | 2 (66.7) | 6 (30.0) | 13 (28.3) |
| Diarrhea | 1 (50.0) | 4 (19.0) | 1 (33.3) | 4 (20.0) | 10 (21.7) |
| Electrocardiogram QT prolonged | 0 | 4 (19.0) | 0 | 6 (30.0) | 10 (21.7) |
| Dizziness | 0 | 5 (23.8) | 2 (66.7) | 2 (10.0) | 9 (19.6) |
| Hot flush | 0 | 4 (19.0) | 2 (66.7) | 3 (15.0) | 9 (19.6) |
| Arthralgia | 0 | 4 (19.0) | 0 | 4 (20.0) | 8 (17.4) |
| Aspartate aminotransferase increased | 0 | 5 (23.8) | 0 | 3 (15.0) | 8 (17.4) |
| Cough | 1 (50.0) | 2 (9.5) | 0 | 5 (25.0) | 8 (17.4) |
| Dyspnea | 0 | 4 (19.0) | 0 | 4 (20.0) | 8 (17.4) |
| Vomiting | 0 | 3 (14.3) | 0 | 5 (25.0) | 8 (17.4) |
| Alanine aminotransferase increased | 0 | 5 (23.8) | 0 | 2 (10.0) | 7 (15.2) |
| Back pain | 0 | 5 (23.8) | 0 | 2 (10.0) | 7 (15.2) |
| Alopecia | 1 (50.0) | 2 (9.5) | 0 | 3 (15.0) | 6 (13.0) |
| Hyponatremia | 0 | 3 (14.3) | 0 | 3 (15.0) | 6 (13.0) |
| Pyrexia | 1 (50.0) | 3 (14.3) | 0 | 2 (10.0) | 6 (13.0) |
| Corona virus infection | 2 (100) | 1 (4.8) | 0 | 2 (10.0) | 5 (10.9) |
| Decreased appetite | 0 | 1 (4.8) | 1 (33.3) | 3 (15.0) | 5 (10.9) |
| Headache | 0 | 3 (14.3) | 0 | 2 (10.0) | 5 (10.9) |
| Hyperglycemia | 0 | 0 | 1 (33.3) | 4 (20.0) | 5 (10.9) |
| Insomnia | 0 | 0 | 1 (33.3) | 4 (20.0) | 5 (10.9) |
| Stomatitis | 1 (50.0) | 4 (19.0) | 0 | 0 | 5 (10.9) |

*Neutropenia was categorized according to the MedDRA preferred terms neutropenia and neutrophil count decreased.
Note:
Treatment-emergent is defined as AE occurring on/after date of first study dose and within 30 days of last study dose.
Note:
For each Preferred Term, patients are counted only once.
Note:
Adverse Events were coded using MedDRA version 22.0.
Data cutoff date: 6 JUN. 2023
Source: BDM TAE02C3: Treatment-emergent Adverse Events ≥10% by Preferred Term (Part C) -All-Treated Analysis Set

All 46 participants (100%) experienced at least one adverse event related either to Compound A or to palbociclib, with neutropenia, fatigue and platelet count decreased as most frequently reported TRAEs (Table).

Thirty-nine (39) of 46 (84.8%) of participants reported a Compound A-treatment related TEAE (Table 6). The most common Compound A treatment-related TEAEs observed in ≥10% of patients (all doses), include fatigue (47.8%), neutropenia (28.3%), constipation (21.7%), Electrocardiogram QT prolonged (19.6%), diarrhea (17.4%), hot flush (15.2%), arthralgia (13%), nausea (13%), platelet count decreased (13%), alopecia (10.9%). Neutropenia was the most common Grade 3 or Grade 4 TRAE and occurred in 47.6% (Grade 3) and 38.1% (Grade 4) of participants, respectively.

The most common palbociclib treatment-related TEAEs observed in ≥10% of patients treated with all doses Compound A and 125 mg Palbociclib include neutropenia in all patients (100%), fatigue (60.9%), platelet count decreased (45.7%), anemia (34.8%) and white blood cell count decreased (26.1%), Constipation (17.4%), Diarrhea (17.4%) and Nausea (17.4%).

Death was reported in a total of 5 (10.9%) out of 46 participants. Of them, 3 (6.5%) were found related to disease under study or complications thereof, and the remaining 2 (4.3%) deaths were reported as related to “other”.

Four patients (8.7%) experienced TEAEs that led to the discontinuation of both Compound A and palbociclib:
One patient dosed with 200 mg Compound A+125 mg palbociclib QD experienced Grade 1 weight loss considered to be related to both Compound Aand palbociclib by the investigator.
One patient dosed with 200 mg Compound A+125 mg Palbociclib QD experienced Grade 1 cough deemed unrelated to Compound A or palbociclib. The patient was taken off study and the discontinuation reason was subject withdrawal.
One patient dosed with 200 mg Compound A+125 mg palbociclib QD experienced Grade 1 dizziness deemed unrelated to Compound Aor palbociclib.
One patient dosed with 500 mg Compound A+125 mg palbociclib QD experienced Grade 3 fatigue that was considered unrelated to Compound A and related to Palbociclib by the investigator. The patient was taken off study and the discontinuation reason was subject withdrawal.

Four patients (8.7%) experienced a TEAE leading to palbociclib discontinuation only:
One patient dosed with 200 mg Compound A+125 mg palbociclib QD experienced Grade 4 neutrophil count decreased considered to be possibly related to both Compound A and Palbociclib by the investigator.
One patient dosed with 200 mg Compound A+125 mg palbociclib QD experienced a Grade 3 cerebral vascular accident deemed unrelated to either Compound A or palbociclib.
One patient dosed with 500 mg Compound A+125 mg palbociclib QD experienced Grade 4 neutrophil count decreased considered related to Palbociclib only by the investigator.
One patient dosed with 500 mg Compound A+125 mg palbociclib QD experienced Grade 3 neutrophil count decreased considered related to palbociclib only by the investigator.

TABLE 5
Compound A or Palbociclib Treatment-related Adverse Events ≥10%
by Preferred Term (Part C)-All-Treated Analysis Set (ARV-471-mBC-101)

| | 180 mg + | 200 mg + | 400 mg + | 500 mg + | |
|---|---|---|---|---|---|
| | Palbo | Palbo | Palbo | Palbo | Total |
| AE Preferred Term [n (%)] | (N = 2) | (N = 21) | (N = 3) | (N = 20) | (N = 46) |
| Any Compound A or Palbociclib | 2 (100) | 21 (100) | 3 (100) | 20 (100) | 46 (100) |
| treatment-related adverse events | | | | | |
| Neutropenia* | 2 (100) | 21 (100) | 3 (100) | 20 (100) | 46 (100) |
| Fatigue | 2 (100) | 13 (61.9) | 1 (33.3) | 12 (60.0) | 28 (60.9) |
| Platelet count decreased | 0 | 11 (52.4) | 2 (66.7) | 10 (50.0) | 23 (50.0) |
| Anemia | 0 | 7 (33.3) | 2 (66.7) | 7 (35.0) | 16 (34.8) |
| White blood cell count decreased | 0 | 5 (23.8) | 2 (66.7) | 5 (25.0) | 12 (26.1) |
| Constipation | 0 | 5 (23.8) | 0 | 6 (30.0) | 11 (23.9) |
| Electrocardiogram QT prolonged | 0 | 4 (19.0) | 0 | 6 (30.0) | 10 (21.7) |
| Diarrhea | 1 (50.0) | 2 (9.5) | 1 (33.3) | 4 (20.0) | 8 (17.4) |
| Nausea | 1 (50.0) | 2 (9.5) | 0 | 5 (25.0) | 8 (17.4) |
| Hot flush | 0 | 2 (9.5) | 2 (66.7) | 3 (15.0) | 7 (15.2) |
| Alopecia | 1 (50.0) | 2 (9.5) | 0 | 3 (15.0) | 6 (13.0) |
| Arthralgia | 0 | 3 (14.3) | 0 | 3 (15.0) | 6 (13.0) |
| Decreased appetite | 0 | 1 (4.8) | 1 (33.3) | 3 (15.0) | 5 (10.9) |
| Vomiting | 0 | 3 (14.3) | 0 | 2 (10.0) | 5 (10.9) |

Note:
Adverse Events were coded using MedDRA version 22.0.
Data cutoff date: 6 JUN. 2023
Source: BDM TAE03C: Compound A or Palbociclib Treatment-related Adverse Events ≥10% by Preferred Term (Part C)-All-Treated Analysis Set

TABLE 6
Compound A Treatment-related Adverse Events in ≥10% of patients
by Preferred Term (Part C)-All-Treated Analysis Set (ARV-471-mBC-101)

| | 180 mg + | 200 mg + | 400 mg + | 500 mg + | |
|---|---|---|---|---|---|
| | Palbo | Palbo | Palbo | Palbo | Total |
| AE Preferred Term [n (%)] | (N = 2) | (N = 21) | (N = 3) | (N = 20) | (N = 46) |
| Any Compound A treatment-related | 2 (100) | 17 (81.0) | 3 (100) | 17 (85.0) | 39 (84.8) |
| adverse events | | | | | |
| Fatigue | 2 (100) | 10 (47.6) | 1 (33.3) | 9 (45.0) | 22 (47.8) |
| Neutropenia* | 2 (100) | 4 (19.0) | 1 (33.3) | 6 (30.0) | 13 (28.3) |
| Constipation | 0 | 5 (23.8) | 0 | 5 (25.0) | 10 (21.7) |
| Electrocardiogram QT prolonged | 0 | 3 (14.3) | 0 | 6 (30.0) | 9 (19.6) |
| Diarrhea | 1 (50.0) | 2 (9.5) | 1 (33.3) | 4 (20.0) | 8 (17.4) |
| Hot flush | 0 | 2 (9.5) | 2 (66.7) | 3 (15.0) | 7 (15.2) |
| Arthralgia | 0 | 3 (14.3) | 0 | 3 (15.0) | 6 (13.0) |
| Nausea | 1 (50.0) | 2 (9.5) | 0 | 3 (15.0) | 6 (13.0) |
| Platelet count decreased | 0 | 3 (14.3) | 1 (33.3) | 2 (10.0) | 6 (13.0) |
| Alopecia | 1 (50.0) | 1 (4.8) | 0 | 3 (15.0) | 5 (10.9) |

Note:
Adverse Events were coded using MedDRA version 22.0.
Data cutoff date: 6 JUN. 2023
Source: BDM TAE03C1: Compound A Treatment-related Adverse Events ≥10% by Preferred Term (Part C)-All-Treated Analysis Set

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

We claim:

1. A method for treating cancer comprising administering to a subject a daily dose of Compound A: 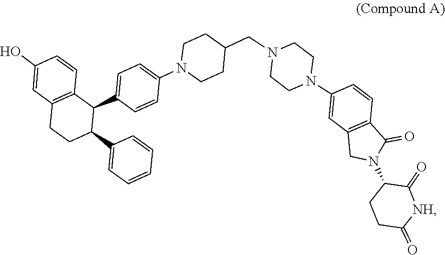 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, in combination with a CDK4/6 inhibitor, wherein the daily dose of Compound A is about 100 mg or 200 mg.

2. The method of claim 1, wherein the CDK4/6 inhibitor is administered concurrently or sequentially.

3. The method of claim 1 or 2, wherein the daily dose of Compound A, or a pharmaceutically acceptable salt thereof, is about 200 mg.

4. The method of claim 1 or 2, wherein the daily dose of Compound A, or a pharmaceutically acceptable salt thereof, is about 100 mg.

5. The method of any one of claims 1 to 4, wherein the daily dose of Compound A, or a pharmaceutically acceptable salt thereof, is administered once per day (QD).

6. The method of any one of claims 1 to 5, wherein the daily dose of Compound A, or a pharmaceutically acceptable salt thereof, is administered orally to the subject.

7. The method of any one of claims 1 to 6, wherein the subject is in a fed state.

8. The method of any one of claims 1 to 7, wherein the CDK4/6 inhibitor is dalpiciclib, palbociclib, lerociclib, AT7519M, dinaciclib, ribociclib, abemaciclib, or palbociclib, or a pharmaceutically acceptable salt thereof.

9. The method of claim 8, wherein the CDK4/6 inhibitor is abemaciclib, ribociclib or palbociclib, or a pharmaceutically acceptable salt thereof.

10. The method of claim 9, wherein the CDK4/6 inhibitor is abemaciclib, or a pharmaceutically acceptable salt thereof.

11. The method of claim 9, wherein the CDK4/6 inhibitor is ribociclib, or a pharmaceutically acceptable salt thereof.

12. The method of claim 9, wherein the CDK4/6 inhibitor is palbociclib, or a pharmaceutically acceptable salt thereof.

13. The method of claim 10, wherein abemaciclib, or a pharmaceutically acceptable salt thereof, is administered to the subject at a dose of 150 mg twice daily.

14. The method of claim 11, wherein Compound A or a pharmaceutically acceptable salt thereof is administered daily in 28-day cycles and ribociclib, or a pharmaceutically acceptable salt thereof, is administered orally at a daily dosage of about 600 mg once daily for 21 days followed by 7 days off treatment for each 28-day cycle.

15. The method of claim 12, wherein Compound A, or a pharmaceutically acceptable salt thereof, is administered daily in 28-day cycles and palbociclib, or a pharmaceutically acceptable salt thereof, is administered orally once a day at 125 mg/day for 21 days followed by 7 days off treatment for each 28-day cycle.

16. The method of claim 1, wherein the daily dose of Compound A, or a pharmaceutically acceptable salt thereof, is about 200 mg in combination with abemaciclib, or a pharmaceutically acceptable salt thereof, which is administered to the subject at a dose of about 150 mg twice daily.

17. The method of claim 1, wherein the daily dose of Compound A or a pharmaceutically acceptable salt thereof about 100 mg, in combination with ribociclib, or a pharmaceutically acceptable salt thereof, which is administered in a daily dose of about 600 mg.

18. The method of any one of claims 1 to 17, wherein the cancer is breast cancer, lung cancer, colon cancer, brain cancer, head and neck cancer, prostate cancer, stomach cancer, pancreatic cancer, ovarian cancer, melanoma, endocrine cancer, uterine cancer, testicular cancer, or bladder cancer.

19. The method of claim 18, wherein the cancer is breast cancer, lung cancer, prostate cancer, pancreatic cancer, or ovarian cancer.

20. The method of claim 19, wherein the cancer is breast cancer, lung cancer, or prostate cancer.

21. The method of claim 20, wherein the cancer is breast cancer.

22. The method of claim 21, wherein the breast cancer is metastatic or locally advanced.

23. The method of claim 21 or 22, wherein the breast cancer is estrogen receptor positive (ER+) breast cancer.

24. The method of claim 23, wherein the estrogen receptor positive (ER+) breast cancer is human epidermal growth factor receptor 2 negative (HER2−).

25. The method of any one of claims 1 to 24, wherein the subject is human.
